SEQUENCE LISTING (73 sequences)

SEQ ID NO: 1 - protein, 25 aa, Artificial
  consensus zinc finger structure
PYKCPECGKSFSQKSDLVKHQRTHT

SEQ ID NO: 2 - protein, 25 aa, Artificial
  consensus zinc finger structure
PYKCSECGKAFSQKSNLTRHQRIHT

SEQ ID NO: 3 - protein, 6 aa, Artificial
  leader peptide
MAEEKP

SEQ ID NO: 4 - protein, 6 aa, Artificial
  leader peptide
MAEERP

SEQ ID NO: 5 - protein, 5 aa, Artificial
  leader peptide
MAERP

SEQ ID NO: 6 - protein, 4 aa, Artificial
  linker
GXKP

SEQ ID NO: 7 - protein, 4 aa, Artificial
  flexible linker
GERP

SEQ ID NO: 8 - protein, 4 aa, Artificial
  flexible linker
GEKP

SEQ ID NO: 9 - protein, 4 aa, Artificial
  flexible linker
GQRP

SEQ ID NO: 10 - protein, 4 aa, Artificial
  flexible linker
GQKP

SEQ ID NO: 11 - protein, 5 aa, Artificial
  linker
GGEKP

SEQ ID NO: 12 - protein, 5 aa, Artificial
  linker
GGQKP

SEQ ID NO: 13 - protein, 5 aa, Artificial
  linker
GSERP

SEQ ID NO: 14 - protein, 7 aa, Artificial
  linker
GGSGEKP

SEQ ID NO: 15 - protein, 7 aa, Artificial
  linker
GGSGQKP

SEQ ID NO: 16 - protein, 8 aa, Artificial
  linker
GGGGSERP

SEQ ID NO: 17 - protein, 10 aa, Artificial
  linker
GGSGGSGEKP

SEQ ID NO: 18 - protein, 10 aa, Artificial
  linker
GGSGGSGQKP

SEQ ID NO: 19 - DNA, 60 bp, Artificial
  oligonucleotide for site-directed mutagenesis
ctcctgcagttggacctgtgccatggccggctgggccgcatagaatggaacaactaaagc

SEQ ID NO: 20 - protein, 220 aa, Artificial
  zinc finger polypeptide
MGEKALPVVYKRYICSFADCGAAYNKNWKLQAHLCKHTGEKPFPCKEEGCEKGFTSLHHLTRHSLTHTGEKNFTCDSDGCDLRFTTKANMKKHFNRFHNIKICVYVCHFENCGKAFKKHNQLKVHQFSHTQQLPYACPVESCDRRFSRSDELTRHIRIHTGQKPFQCRICMRNFSRSDHLTTHIRTHTGEKPFACDICGRKFARSDERKRHTKIHLRQKD

SEQ ID NO: 21 - DNA, 27 bp, Artificial
  zinc finger
gcgtgggcgtgtacctggatgggagac

SEQ ID NO: 22 - protein, 85 aa, Artificial
  SV40-NLS-VP64-c-myc
AARNSGPKKKRKVELQLTSDALDDFDLDMLGSDALDDFDLDMLGSDALDDFDLDMLGSDALDDFDLDMLSSQLSQEQKLISEEDL

SEQ ID NO: 23 - DNA, 9 bp, Artificial
  zinc finger binding domain
gcggnnnnn

SEQ ID NO: 24 - DNA, 9 bp, Artificial
  zinc finger binding domain
nnnnnggcg

SEQ ID NO: 25 - DNA, 32 bp, Mus musculus
  misc_binding (8)..(25)
agtggtgttaagtgggtttggggcgccaagct

SEQ ID NO: 26 - protein, 7 aa, Artificial
  alpha helical region of TNFR1-M4-2
RSADLTR

SEQ ID NO: 27 - protein, 7 aa, Artificial
  alpha helical region of TNFR1-M4-2
RRDHLSE

SEQ ID NO: 28 - protein, 7 aa, Artificial
  alpha helical region of TNFR1-M4-2
TNDSRTN

SEQ ID NO: 29 - protein, 7 aa, Artificial
  alpha helical region of TNFR1-M4-2
RSQHLTE

SEQ ID NO: 30 - protein, 7 aa, Artificial
  alpha helical region of TNFR1-M4-2
TSSHLSK

SEQ ID NO: 31 - protein, 7 aa, Artificial
  alpha helical region of TNFR1-M4-2
QSNARKT

SEQ ID NO: 32 - protein, 322 aa, Artificial
  zinc finger chimeric repressor
MAERPYACPVESCDRRFSRSADLTRHIRIHTGQKPFQCRICMRNFSRRDHLSEHIRTHTGEKPFACDICGRKFATNDSRTNHTKIHTGSERPYACPVESCDRRFSRSQHLTEHIRIHTGQKPFQCRICMRNFSTSSHLSKHIRTHTGEKPFACDICGRKFAQSNARKTHTKIHLRQKDAARNSGPKKKRKVDGGGALSPQHSAVTQGSIIKNKEGMDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVEQKLISEEDL

SEQ ID NO: 33 - DNA, 31 bp, Mus musculus
  misc_binding (9)..(26)
cccccagtgaggggctgggggtgtggctcag

SEQ ID NO: 34 - protein, 6 aa, Artificial
  linker
TGSERP

SEQ ID NO: 35 - protein, 7 aa, Artificial
  EPO-M10-9
RSSHLST

SEQ ID NO: 36 - protein, 7 aa, Artificial
  EPO-M10-9
RSDTLTR

SEQ ID NO: 37 - protein, 7 aa, Artificial
  EPO-M10-9
RNDHRTK

SEQ ID NO: 38 - protein, 7 aa, Artificial
  EPO-M10-9
RSDALSE

SEQ ID NO: 39 - protein, 7 aa, Artificial
  EPO-M10-9
RNSHRTK

SEQ ID NO: 40 - protein, 7 aa, Artificial
  EPO-M10-9
RSDNLTR

SEQ ID NO: 41 - protein, 263 aa, Artificial
  transcriptional activation peptide
MAERPYACPVESCDRRFSRSADLTRHIRIHTGQKPFQCRICMRNFSRRDHLSEHIRTHTGEKPFACDICGRKFATNDSRTNHTKIHTGSERPYACPVESCDRRFSRSQHLTEHIRIHTGQKPFQCRICMRNFSTSSHLSKHIRTHTGEKPFACDICGRKFAQSNARKTHTKIHLRQKDAARNSGPKKKRKVELQLTSDALDDFDLDMLGSDALDDFDLDMLGSDALDDFDLDMLGSDALDDFDLDMLSSQLSQEQKLISEEDL

SEQ ID NO: 42 - DNA, 38 bp, Artificial
  TFIIIAZif binding site with Primer A
tcgactatgcgtgggcgtgtacctggatgggagaccgg

SEQ ID NO: 43 - DNA, 38 bp, Artificial
  TFIIIAZif binding site with Primer D
cgcgccggtctcccatccaggtacacgcacccgcatag

SEQ ID NO: 44 - DNA, 36 bp, Artificial
  primer
cataacttcgtataatgtatgctatacgaagttatt

SEQ ID NO: 45 - DNA, 44 bp, Artificial
  primer
ctagaataacttcgtatagcatacattatacgaagttatgagct

SEQ ID NO: 46 - DNA, 34 bp, Artificial
  PCR anealing sequence
ctacgcccatgggaggagagaaaggcgctgccgg

SEQ ID NO: 47 - DNA, 73 bp, Artificial
  PCR anealing sequence
ctagcagtactcataacttcgtatagcatacattatacgaagttatccagaatagaatgacacctactcagac

SEQ ID NO: 48 - DNA, 68 bp, Artificial
  primer
ctacgcctcgagataacttcgtataatgtatgctatacgaagttatggatcctctcgcctcggcctcc

SEQ ID NO: 49 - DNA, 30 bp, Artificial
  primer
tacctaccatggtggtcagactctccgggg

SEQ ID NO: 50 - DNA, 34 bp, Artificial
  primer
gactatgcgcgcgtacccgggtcgagtaggcgtg

SEQ ID NO: 51 - DNA, 36 bp, Artificial
  primer
taggctagtactcacacctccccctgaacctgaaac

SEQ ID NO: 52 - DNA, 38 bp, Artificial
  with primer 0
tcgagtatgcgtgggcgtgtacctggatgggagaccgg

SEQ ID NO: 53 - DNA, 38 bp, Artificial
  with Primer R
cgcgccggtctcccatccaggtacacgcacccgcatac

SEQ ID NO: 54 - DNA, 21 bp, Artificial
  primer
ccagcctgagtgcaaaattca

SEQ ID NO: 55 - DNA, 20 bp, Artificial
  primer
caggctcgacactggattcc

SEQ ID NO: 56 - DNA, 25 bp, Artificial
  primer
tgctgactttgttccctgctgtgca

SEQ ID NO: 57 - DNA, 20 bp, Artificial
  primer
agcaaagaccccaacgagaa

SEQ ID NO: 58 - DNA, 15 bp, Artificial
  primer
ggcggcggtcacgaa

SEQ ID NO: 59 - DNA, 22 bp, Artificial
  primer
cgcgatcacatggtcctgctgg

SEQ ID NO: 60 - protein, 48 aa, Artificial
  TNFR1-M4-2
RSADLTRRRDHLSETNDSRTNTGSERPRSQHLTETSSHLSKQSNARKT

SEQ ID NO: 61 - DNA, 70 bp, Artificial
  TFIIIAZif binding site with Primer B
tcgactatgcgtgggcgtgtacctggatgggagaccgtatgcgtgggcgtgtacctggatgggagaccgg

SEQ ID NO: 62 - DNA, 102 bp, Artificial
  TFIIIAZif binding site with Primer C
tcgactatgcgtgggcgtgtacctggatgggagaccgtatgcgtgggcgtgtacctggatgggagaccgtatgcgtgggcgtgtacctggatgggagaccgg

SEQ ID NO: 63 - DNA, 70 bp, Artificial
  TFIIIAZif binding site with Primer E
cgcgccggtctcccatccaggtacacgcacccgcatacggtctcccatccaggtacacgcacccgcatag

SEQ ID NO: 64 - DNA, 102 bp, Artificial
  TFIIIAZif binding site with Primer F
cgcgccggtctcccatccaggtacacgcacccgcatacggtctcccatccaggtacacgcacccgcatacggtctcccatccaggtacacgcacccgcatag

SEQ ID NO: 65 - DNA, 70 bp, Artificial
  with Primer P
tcgagtatgcgtgggcgtgtacctggatgggagaccgtatgcgtgggcgtgtacctggatgggagaccgg

SEQ ID NO: 66 - DNA, 102 bp, Artificial
  with primer Q
tcgagtatgcgtgggcgtgtacctggatgggagaccgtatgcgtgggcgtgtacctggatgggagaccgtatgcgtgggcgtgtacctggatgggagaccgg

SEQ ID NO: 67 - DNA, 70 bp, Artificial
  with primer S
cgcgccggtctcccatccaggtacacgcacccgcatacggtctcccatccaggtacacgcacccgcatac

SEQ ID NO: 68 - DNA, 102 bp, Artificial
  with Primer T
cgcgccggtctcccatccaggtacacgcacccgcatacggtctcccatccaggtacacgcacccgcatacggtctcccatccaggtacacgcacccgcatac

SEQ ID NO: 69 - protein, 31 aa, Artificial
  preferred zinc finger framework
XXCXXXXXCXXXXXXXXXXXXXXHXXXXXXX

SEQ ID NO: 70 - protein, 31 aa, Artificial; identical to SEQ ID NO: 69
  zinc finger framework A′

SEQ ID NO: 71 - protein, 24 aa, Artificial
  zinc finger motif
XCXXXXCXXXFXXXXXLXXHXXXH

SEQ ID NO: 72 - protein, 4 aa, Artificial
  linker
TGXX

SEQ ID NO: 73 - protein, 5 aa, Artificial
  linker
TGXXP

(19) United States
(12) Patent Application Publication
(10) Pub. No.: US 2005/0235369 A1
(43) Pub. Date: Oct. 20, 2005

(54) Gene regulation II

(76) Inventors: Yen Choo, London (GB); John Girdlestone, Cambridge (GB); Michael Moore, London (GB)

Correspondence Address: ROBINS & PASTERNAK, 1731 EMBARCADERO ROAD, SUITE 230, PALO ALTO, CA, 94303, US

(21) Appl. No.: 10/473,238

(22) PCT Filed: Mar. 28, 2002
(86) PCT No.: PCT/US02/09703

§ 371 Date: May 9, 2005

(30) Foreign Application Priority Data
Mar. 28, 2001 (GB) 0107757.7

(51) Int. Cl. A01K 67/027

(52) U.S. Cl. 800/14

(57) ABSTRACT

We describe a transgenic non-human animal comprising a heterologous nucleic acid binding polypeptide which binds to a target gene and modulates its expression, in which the heterologous nucleic acid binding polypeptide is encoded by a transgene, and in which the expression of a target gene in at least one cell is modulated compared to a non-transgenic animal.

### FIELD OF THE INVENTION

This invention relates to the field of gene regulation. In particular, we describe methods of regulating the expression of genes in non-human transgenic animals, as well as gene therapy.

### BACKGROUND OF THE INVENTION

Transgenic animals have been widely used to study the relationship between genetics and disease in animal models, and the effects of therapeutic treatments for these diseases. Transgenic technology has also been employed in the creation of transgenic livestock, for improvement of animal products, or for the large-scale production of useful biological products.

Transgenic animal models have proved to be extremely powerful for the study of developmental processes. However, due to inherent problems in the original protocols for producing transgenic animals, the technique has not yet been as generally useful for studying processes in mature animals. Originally, gene targeting involved insertion of nucleic acid into the desired position in the gene or genome, by homologous recombination in animal cells. This procedure of transgenesis enabled the study of “loss of function” or “gain of function” mutations. These are referred to as knock-out and knock-in models, respectively. Both of these systems, although extremely valuable in some circumstances have different and potentially significant problems associated with them.

Knock-but mutations are created by disruption of a portion or the whole of a target gene, creating a null allele. To generate a homozygous animal lacking an active copy of the gene, null allele animals must be cross bred and the required progeny selected. There are two main drawbacks of this technology, embryonic lethality and developmental compensation. Animals derived from this procedure are affected by target gene dysfunction throughout ontogenesis. Embryonic lethality may result if the gene plays a central role in development. This is not always a fair reflection on the therapeutic potential of such genes because a gene that is vital during development may not be required for viability of mature animals. For example, the endothelins-1 and -3 (ET-1 and ET-3) have been implicated in the regulation of blood pressure. However, the role of these proteins could not be assessed in mature mice, as homozygous ET-1 or ET-3 knock-out mice die at birth (Baynash, A. et al., Cell 79: 1277-1285 (1994); Kurihara, Y. et al., Nature 368: 703-710 (1994) and Yanagisawa, M. et al., Proc. Natl. Acad. Sci. USA 85: 6964-6967 (1988)). Developmental compensation is a phenomenon whereby a missing gene function is compensated for, during the course of development, by a related gene product. This may not normally be possible in a mature animal, and may mask the true role of the targeted gene in mature animals.

A knock-in transgenic animal is created by the addition of either an exogenous or an endogenous cDNA or gDNA to a cell. The main drawbacks of this procedure are usually due to the size of the cDNA or gDNA fragment that has to be delivered to the host cell, and the reliance of gene expression on a suitable point of recombination. Often, transgenes are not expressed because they have integrated into a transcriptionally inactive region of the genome.

A further problem relevant to both the basic procedures above is that the mutant gene is present in every cell of the transgenic animal. Therefore, it is not possible to study the biological function of a particular gene in a specific cell type, and any relevant data may be masked by the effects of the genetic modification throughout the animal. Many of the problems associated with such transgenic systems are being addressed by recent advances in targeted gene delivery, tissue specific gene expression, inducible gene expression and site-specific recombination. However, even the most advanced procedures using site-specific recombinases suffer from chimerism due to incomplete activation of the recombinase in all cells.

### SUMMARY OF THE INVENTION

Our invention is based on the demonstration, for the first time, that a transgenic animal can be created which expresses a nucleic acid binding polypeptide from a transgene. We show for the first time that the nucleic acid binding polypeptide binds to and modulates the expression of a gene in the animal. We show that both up-regulation as well as down-regulation can be achieved, of both endogenous and heterologous genes.

According to a first aspect of the present invention, we provide a transgenic non-human animal comprising a heterologous nucleic acid binding polypeptide which binds to a target gene and modulates its expression, in which the heterologous nucleic acid binding polypeptide is encoded by a transgene, and in which the expression of a target gene in at least one cell is modulated compared to a non-transgenic animal.

There is provided, according to a second aspect of the present invention, a method of modulating the expression of a target gene in a transgenic animal, the method comprising the steps of: (a) providing a transgenic animal comprising a transgene which expresses a heterologous nucleic acid binding polypeptide; and (b) allowing the nucleic acid binding polypeptide to bind to a target gene, thereby modulating the expression of the target gene.

Preferably, the expression of an endogenous gene is modulated. Alternatively or in addition, the expression of a heterologous gene may be modulated. Thus, the gene whose expression is modulated may comprise a heterologous gene which is introduced into the cell or an ancestor of that cell. Preferably, the nucleic acid binding polypeptide binds to a promoter or other control sequence of a gene to modulate its expression. More preferably, the gene whose expression is modulated comprises erythropoietin (EPO) or TNF receptor 1 (TNFR1).

The transgenic animal or method may be such that modulation of expression of the gene occurs in a subset of cells of the transgenic animal. Preferably, the subset of cells comprises cells of a similar tissue type, location or developmental stage. Alternatively, modulation of expression of the gene occurs in substantially all cells of the transgenic animal.

In a highly preferred embodiment of the invention, the nucleic acid binding polypeptide comprises a zinc finger polypeptide. The nucleic acid binding polypeptide may further comprise a transcriptional effector domain. The transcriptional effector domain may comprise a transcriptional repressor domain selected from the group consisting of: a KRAB-A domain, an engrailed domain and a snag domain. Alternatively, or in addition, the transcriptional effector domain may comprise a transcriptional activation domain selected from the group consisting of: VP16, VP64, transactivation domain 1 of the p65 subunit (RelA) of nuclear factor-κB, transactivation domain 2 of the p65 subunit (RelA) of nuclear factor-κB, and the activation domain of CTCF.

In a preferred embodiment, the nucleic acid binding polypeptide comprises a sequence which is selected from the group consisting of: TNFR1-M4-2, TNFR1-M4-2-Kox1, EPO-M10-9 and EPO-M10-9-VP64.

The nucleic acid binding polypeptide may be selected by phage display. Alternatively, or in addition, the nucleic acid binding polypeptide may be engineered by rational design. In a preferred embodiment of the invention, expression of the target gene is downregulated by 10%, 20%, 30%, 40%, 50%, 60%, 70%, 80% 90% or more. In a highly preferred embodiment of the invention, expression of the target gene is downregulated by at least 80% compared to a non-transgenic animal.

We provide, according to a third aspect of the present invention, a transgenic non-human animal comprising stably integrated into the genome of the animal a nucleotide sequence encoding a nucleic acid binding polypeptide operably linked to a promoter, in which the nucleic acid binding polypeptide is expressed in at least one cell of the transgenic animal, and in which the expression of a target gene is modulated by virtue of the nucleic acid binding polypeptide binding to the target gene.

As a fourth aspect of the present invention, there is provided a method of producing a transgenic animal comprising a heterologous nucleic acid binding polypeptide, the method comprising the steps of: (a) providing a nucleic acid sequence encoding a heterologous nucleic acid binding polypeptide, in which the nucleic acid binding polypeptide binds to and regulates the expression of a gene; and (b) introducing the nucleic acid sequence into the animal in such a manner that the nucleic acid sequence is stably integrated into the genome of the animal.

Preferably, the method is such that the nucleic acid sequence is introduced into a cell, the cell being implanted into an animal or an embryo of the animal.

We provide, according to a fifth aspect of the present invention, a method of determining the function of a gene, the method comprising the steps of: (a) providing a transgenic animal comprising a heterologous nucleic acid binding polypeptide which binds to a target gene and modulates its expression; and (b) observing a phenotype of the transgenic animal.

The present invention, in a sixth aspect, provides a method of identifying a gene of interest, the method comprising the steps of: (a) providing a transgenic animal comprising a heterologous nucleic acid binding polypeptide which binds to a first target gene and modulates its expression; and (b) detecting modulation of expression of a second gene by the transgenic animal.

In a seventh aspect of the present invention, there is provided a gene identified by a method according to the sixth aspect of the invention.

According to an eighth aspect of the present invention, we provide a method of differential screening of a gene, the method comprising steps (a) and (b) according to the sixth aspect of the invention.

We provide, according to a ninth aspect of the invention, a method of identifying a molecule which modulates the interaction between a nucleic acid binding polypeptide and a target nucleic acid sequence, the method comprising the steps of: (a) providing a transgenic animal comprising a heterologous nucleic acid binding polypeptide which is capable of binding to a target gene and modulates its expression, in which the heterologous nucleic acid binding polypeptide is encoded by a transgene; (b) exposing one or more of the transgenic animal, the nucleic acid binding polypeptide and the target nucleic acid sequence to a candidate molecule; and (c) detecting binding or modulation of binding between the nucleic acid binding polypeptide and the target nucleic acid sequence.

Preferably, binding between the nucleic acid binding polypeptide and the target nucleic acid sequence is detected by detecting expression of the target nucleic acid sequence, or by detecting expression of a nucleic acid sequence linked to the target nucleic acid sequence. Moreover, binding between the nucleic acid binding polypeptide and the target nucleic acid sequence may be detected by observing a visible phenotype.

There is provided, in accordance with a tenth aspect of the present invention, a molecule identified by a method according to the ninth aspect of the invention.

As an eleventh aspect of the invention, we provide a method of modulating the interaction between a nucleic acid binding polypeptide and a target nucleic acid sequence in a system, the method comprising exposing the system or any of its components to a molecule according to the ninth aspect of the invention.

We provide, according to a twelfth aspect of the invention, there is provided a method of producing a polypeptide, the method comprising the steps of: (a) providing a transgenic animal comprising a heterologous nucleic acid binding polypeptide which is encoded by a transgene, and a nucleic acid sequence encoding a polypeptide, in which the nucleic acid binding polypeptide binds to a target nucleic acid sequence to up-regulate the expression of the polypeptide; and (b) harvesting the polypeptide from the transgenic animal.

The polypeptide is preferably secreted into the mammary or other fluid of the animal, and in which the polypeptide is isolated from the fluid.

According to a thirteenth aspect of the present invention, we provide a polypeptide produced by a method according to the twelth aspect of the invention.

### BRIEF DESCRIPTION OF THE DRAWINGS

FIG. 1 shows the gene cassettes for the specific expression of zinc finger polypeptides in T-cells. The exons of human CD2 (hCD2) are shown by the numbers within the boxes. The cassette displayed refers to the constructs MITFIIIAZif, MITNFR1 and MIEPO. The horizontal arrow indicates the direction of transcription. Restriction sites for construction of the cassette are shown by arrows. The diagram is not to scale.

FIG. 2 shows the gene cassettes for the human CD2 (hCD2) reporter constructs used in T-cells. Part (a) shows the MICD2 cassette, and part (b) shows the MI4CD2 cassette. The exons of hCD2 are indicated by the numbers inside the boxes. The box labelled TFIIIAZif indicates the position of the TFIIIAZif binding sites (which can be in 1 to 3 copies and in either orientation), for the specific expression of the reporter by the TFIIIAZif-NLS-VP64-cmyc activator peptide. The direction of transcription is indicated by horizontal arrows. Restriction sites used in the construction are shown by arrows. The diagram is not to scale.

FIG. 3 shows the combined reporter and expression cassette for specific use in B-cells. The TFIIIAZif-NLS-VP64-cmyc chimeric peptide is expressed from the B-cell specific promoter of the human CD19 (hCD19) gene. TFIIIAZif-NLS-VP64-cmyc then activates transcription of the reporter gene (destabilised enhanced green fluorescent protein) by binding to the TFIIIAZif binding sites upstream of the reporter gene. The TFIIIAZif binding sites may be in 1, 2, or 3 copies (and in either orientation), and are indicated by the box labelled, TFIIIAZif. The horizontal arrows indicate the direction of transcription of each gene. The positions of restriction sites used for construction of the cassette are shown. The diagram is not to scale.

### DETAILED DESCRIPTION OF THE INVENTION

Although it has been suggested previously that vectors comprising sequences encoding nucleic acid binding polypeptides may be used for expression in transgenic animals (WO 00/73434 and WO01/00815), these documents do not demonstrate that modulation of gene activity may be achieved. Furthermore, neither WO 00/73434 nor WO01/00815 discloses the construction of a transgenic animal expressing a nucleic acid binding polypeptide, nor do they disclose or suggest which genes may be targetted. Each of these are demonstrated for the first time in this document.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). The practice of the present invention will employ, unless otherwise indicated, conventional techniques of chemistry, molecular biology, microbiology, recombinant DNA, immunology, chemical methods, pharmaceutical formulations and delivery and treatment of patients, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; and, D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

Transgenic Animals

A transgenic animal is an animal, preferably a non-human animal, containing at least one foreign gene, called a transgene, in its genetic material. Preferably, the transgene is contained in the animal's germ line such that it can be transmitted to the animal's offspring. Transgenic animals may carry the transgene in all their cells or may be genetically mosaic.

According to a method of conventional transgenesis, copies of normal or modified genes are injected into the male pronucleus of the zygote and become integrated into the genomic DNA of the recipient animal. The transgene is transmitted in a Mendelian manner in established transgenic strains.

Constructs useful for creating transgenic animals useful according to the invention comprise genes encoding nucleic acid binding polypeptides, optionally under the control of nucleic acid sequences directing their expression in cells of a particular lineage. Alternatively, nucleic acid binding polypeptide encoding constructs may be under the control of their native promoters, or inducibly regulated. Typically, DNA fragments on the order of 10 kilobases or less are used to construct a transgenic animal (Reeves, 1998, New. Anat., 253:19). A transgenic animal expressing one transgene can be crossed to a second transgenic animal expressing a second transgene such that their offspring will carry both transgenes.

Although the majority of studies have involved transgenic mice, other species of transgenic animal have also been produced, such as rabbits, sheep, pigs (Hammer et al., 1985, Nature 315:680-683; Kumar, et al., U.S. Pat. No. 0,592,2854; Seebach, et al., U.S. Pat. No. 0,603,0833) and chickens (Salter et al., 1987, Virology 157:236-240). While the transgenic animals described in the present invention are not limited to mice, the description which follows details the methodology for transgene expression in smaller animals, such as mice, but may be adapted for larger animals (for example, sheep and pigs) as need requires. Transgenic animals are currently being developed to serve as bioreactors for the production of useful pharmaceutical compounds (Van Brunt, 1988, Bio/Technology 6:1149-1154; Wilmut et al., 1988, New Scientist (July 7 issue) pp. 56-59). Up-regulation of genes expressing useful polypeptides, such as therapeutic polypeptides, by means of a heterologous nucleic acid binding polypeptide, may be used to produce such polypeptides in transgenic animals. Preferably, the polypeptides are secreted into an extratable fluid, such as blood or mammary fluid (milk), to enable easy isolation of the polypeptide.

Transgenic animals comprising transgenes, optionally integrated within the genome, and expressing heterologous zinc finger and other nucleic acid binding polypeptides from transegenes, may be created by a variety of methods. Methods for producing transgenic animals are known in the art, and are described by Gordon, J. & Ruddle, F. H. Science 214: 1244-1246 (1981); Jaenisch, R. Proc. Natl. Acad. Sci. USA 73: 1260-1264 (1976); Gossler et al., (1986); Hogan et al., Manipulating the Mouse Embryo: A Laboratory Manual, (1988); and U.S. Pat. Nos. 5,175,384; 5,434,340 and 5,591,669. Further methods and techniques for producing transgenic animals may be found in the Examples. The transgenic animal is preferably selected from the group consisting of: mouse, rat, sheep, goat, pig and cow.

Mice have become the main species used in the field of transgenic animals for a number of reasons, which include, their small size, low cost, short generation time and fairly well defined genetics. There are several principal methods used to create such transgenic animals, such as DNA microinjection and retrovirus-mediated gene transfer. These methods may also be used equally to produce transgenic animals of other species and genera.

DNA microinjection is described in detail in Gordon, J. & Ruddle, F. H. Science 214: 1244-1246 (1981), and was the first technique which proved to be generally successful in mammals. The procedure involves the direct injection of a gene (or multiple gene) construct into the pronucleus of a fertilized ovum. The fertilized ovum is then transferred into the oviduct of a recipient female. The insertion of DNA by this mechanism is a random process and there is no guarantee that the genes will be expressed from their point of recombination. The DNA construct may also be injected into an in vitro culture of cells, to enable insertion of the desired DNA by homologous recombination. Introduction of these cells into an embryo at the blastocyst stage results in a chimeric animal.

Retrovirus-mediated gene transfer uses a viral vector to deliver heterologous genes into a cell. Again, the result is a chimeric animal. With procedures that result in chimeric animals, the progeny must be cross-bred to generate fully homozygous animals and so the procedure can be very labour intensive.

### Production of Transgenic Animals by Microinjection of Oocytes

A detailed description of production of a transgenic animal expressing a nucleic acid binding polypeptide, by micro-injection of oocytes, is provided here.

In preferred embodiments the transgenic animals described here are produced by i) microinjecting a recombinant nucleic acid molecule encoding a nucleic acid binding polypeptide into a fertilized egg to produce a genetically altered egg; ii) implanting the genetically altered egg into a host female animal of the same species; iii) maintaining the host female for a time period equal to a substantial portion of the gestation period of said animal fetus. iv) harvesting a transgenic animal having at least one cell that has developed from the genetically altered mammalian egg, which expresses a gene which encodes a nucleic acid binding polypeptide.

In general, the use of microinjection protocols in transgenic animal production is typically divided into four main phases: (a) preparation of the animals; (b) recovery and maintenance in vitro of one or two-celled zygotes, fertilised eggs or embryos; (c) microinjection of the zygotes, embryos etc and (d) reimplantation of zygotes, embryos etc into recipient females. The methods used for producing transgenic livestock, do not differ in principle from those used to produce transgenic mice. Compare, for example, Gordon et al. (1983) Methods in Enzymology 101:411, and Gordon et al. (1980) PNAS 77:7380 concerning, generally, transgenic mice with Hammer et al. (1985) Nature 315:680, Hammer et al. (1986) J Anim Sci 63:269-278, Wall et al. (1985) Biol Reprod. 32:645-651, Pursel et al. (1989) Science 244:1281-1288, Vize et al. (1988) J Cell Science 90:295-300, Muller et al. (1992) Gene 121:263-270, and Velander et al (1992) PNAS 89:12003-12007, each of which teach techniques for generating transgenic swine. See also, PCT Publication WO 90/03432, and PCT Publication WO 92/22646 and references cited therein.

One step of the preparatory phase comprises synchronizing the estrus cycle of at least the donor females, and inducing superovulation in the donor females prior to mating. Superovulation typically involves administering drugs at an appropriate stage of the estrus cycle to stimulate follicular development, followed by treatment with drugs to synchronize estrus and initiate ovulation. As described in the example below, a pregnant female animal's serum is typically used to mimic the follicle-stimulating hormone (FSH) in combination with human chorionic gonadotropin (hCG) to mimic luteinizing hormone (LH). The efficient induction of superovulation depends, as is well known, on several variables including the age and weight of the females, and the dose and timing of the gonadotropin administration. See for example, Wall et al. (1985) Biol. Reprod. 32:645, describing superovulation of pigs. Superovulation increases the likelihood that a large number of healthy embryos will be available after mating, and further allows the practitioner to control the timing of experiments

After mating, one or two-cell fertilized eggs from the superovulated females are harvested for microinjection. A variety of protocols useful in collecting eggs from animals are known. For example, in one approach, oviducts of fertilized superovulated females can be surgically removed and isolated in a buffer solution/culture medium, and fertilized eggs expressed from the isolated oviductal tissues. See, Gordon et al. (1980) PNAS 77:7380; and Gordon et al. (1983) Methods in Enzymology 101:411. Alternatively, the oviducts can be cannulated and the fertilized eggs can be surgically collected from anesthetized animals by flushing with buffer solution/culture medium, thereby eliminating the need to sacrifice the animal. See Hammer et al. (1985) Nature 315:600. The timing of the embryo harvest after mating of the superovulated females can depend on the length of the fertilization process and the time required for adequate enlargement of the pronuclei. This temporal waiting period can range from, for example, up to 48 hours for larger animal species. Fertilized eggs appropriate for microinjection, such as one-cell ova containing pronuclei, or two-cell embryos, can be readily identified under a dissecting microscope

The equipment and reagents needed for microinjection of the isolated embryos from larger animals are similar to that used for the mouse. See, for example, Gordon et al. (1983) Methods in Enzymology 101:411; and Gordon et al. (1980) PNAS 77:7380, describing equipment and reagents for microinjecting embryos. Briefly, fertilized eggs are positioned with an egg holder (fabricated from 1 mm glass tubing), which is attached to a micro-manipulator, which is in turn coordinated with a dissecting microscope optionally fitted with differential interference contrast optics. Where visualization of pronuclei is difficult because of optically dense cytoplasmic material, such as is generally the case with swine embryos, centrifugation of the embryos can be carried out without compromising embryo viability. Wall et al. (1985) Biol. Reprod. 32:645. Centrifugation will usually be necessary in this method. A recombinant nucleic acid molecule encoding a nucleic acid binding polypeptide is provided, typically in linearized form, by linearizing the recombinant nucleic acid molecule with at least 1 restriction endonuclease, with an end goal being removal of any prokaryotic sequences as well as any unnecessary flanking sequences. In addition, a recombinant nucleic acid molecule containing a tissue specific promoter and the human class I gene may be isolated from the vector sequences using 1 or more restriction endonucleases. Techniques for manipulating and linearizing recombinant nucleic acid molecules are well known and include the techniques described in Molecular Cloning: A Laboratory Manual, Second Edition. Maniatis et al. eds., Cold Spring Harbor, N.Y. (1989). The linearized recombinant nucleic acid molecule may be microinjected into an egg to produce a genetically altered mammalian egg using well known techniques. Typically, the linearized nucleic acid molecule is microinjected directly into the pronuclei of the fertilized eggs as has been described by Gordon et al. (1980) PNAS 77:7380-7384. This leads to the stable chromosomal integration of the recombinant nucleic acid molecule in a significant population of the surviving embryos. See for example, Brinster et al. (1985) PNAS 82:4438-4442 and Hammer et al. (1985) Nature 315:600-603. The microneedles used for injection, like the egg holder, can also be pulled from glass tubing. The tip of a microneedle is allowed to fill with plasmid suspension by capillary action. By microscopic visualization, the microneedle is then inserted into the pronucleus of a cell held by the egg holder, and plasmid suspension injected into the pronucleus. If injection is successful, the pronucleus will generally swell noticeably. The microneedle is then withdrawn, and cells which survive the microinjection (e.g. those which do not lyse) are subsequently used for implantation in a host female.

The genetically altered animal embryo is then transferred to the oviduct or uterine horns of the recipient. Microinjected embryos are collected in the implantation pipette, the pipette inserted into the surgically exposed oviduct of a recipient female, and the microinjected eggs expelled into the oviduct. After withdrawal of the implantation pipette, any surgical incision can be closed, and the embryos allowed to continue gestation in the foster mother. See, for example, Gordon et al. (1983) Methods in Enzymology 101:411; Gordon et al. (1980) PNAS 77:7390; Hammer et al. (1985) Nature 315:600; and Wall et al. (1985) Biol. Reprod. 32:645

The host female mammals containing the implanted genetically altered mammalian eggs are maintained for a sufficient time period to give birth to a transgenic mammal having at least 1 cell which expresses the recombinant nucleic acid molecule of the present invention that has developed from the genetically altered mammalian egg.

At two-four weeks of age (post-natal), tissue samples are taken from the transgenic offspring and digested with Proteinase K. DNA from the samples is phenol-chloroform extracted, then digested with various restriction enzymes. The DNA digests are electrophoresed on a Tris-borate gel, blotted on nitrocellulose, and hybridized with a probe consisting of the at least a portion of the coding region of the recombinant cDNA of interest (i.e., a nucleic acid encoding a nucleic acid binding polypeptide such as a zinc finger polypeptide) which had been labeled by extension of random hexamers. Under conditions of high stringency, this probe should not hybridize with the endogenous (non-transgene) genes, but should produce a hybridization signal in animals expressing the transgene, allowing for the identification of transgenic pigs.

The present invention provides many advantages over the prior art. The use of nucleic acid binding polypeptides such as zinc finger polypeptides to regulate the expression of genes within transgenic animals (as described here) overcomes many of the usual difficulties in creating transgenic animals. For example, there is no need for the introduction of large gDNA sequences. Expression of the nucleic acid binding polypeptide (for example, zinc finger) may be induced at any stage during development by the use of inducible expression systems. Gene knock-out or over-expression does not need to be permanent, i.e. target gene activation or repression is reversible using a zinc finger polypeptide or other nucleic acid binding polypeptide. Degrees of gene expression or repression can be achieved, rather than the all-or-nothing approach using gene deletion or addition. Zinc finger polypeptides and other nucleic acid binding polypeptides act in trans to regulate gene expression. Thus, there is no need to create a homozygous animal, and this can save both time and money in the preparation of new transgenic animals.

Gene Regulation

The present invention demonstrates for the first time the specific regulation of the expression of a gene in an animal, in particular a transgenic animal, with the use of nucleic acid binding polypeptides. In particular, we show regulation or modulation of expression of an endogenous gene in a transgenic animal. We describe zinc finger polypeptides that have been engineered, by rational design or selection, or by a combination of both, to bind any nucleotide sequence within an animal or animal cell. The target nucleotide sequence may be any nucleotide sequence. For example, it may be a nucleotide sequence which is associated with a gene of the animal, an integrated virus, a nucleotide sequence that has been deliberately introduced, or an RNA transcript. Expression of such heterologous nucleic acid binding polypeptides in the cells of the transgenic animal enables modulation (e.g., up-regulation and down-regulation) of expression of a gene or other nucleic acid sequence of interest to be achieved.

The modulation of gene expression may comprise up-regulation or down-regulation. Methods of assaying the level of expression of a gene are known in the art, and include reporter assays (such as CAT assays), ELISA assays, FRET (fluorescence resonance energy transfer), luciferase assays, etc. Gene expression is however most easily measured by assaying the expression of a reporter gene.

The reporter gene may encode an enzyme capable of catalysing an enzymatic reaction with a detectable end-point. Alternatively, the reporter gene may encode a molecule capable of regulating cell growth, such as providing a required nutrient. Preferably, the reporter gene encodes Green Fluorescent Protein (GFP), luciferase, β-galactosidase, or chloramphenicol acetyl transferase (CAT).

The enzymatic activity may be luminescence inducing activity. “Luminescence” refers to the production of light or other radiation by a chemical reaction, and includes bioluminescence or chemiluminescence. Preferably, the luminescence inducing activity is preferably provided by luciferase.

The signal may be emission or absorption of electromagnetic radiation, for example, light. Preferably, the signal is a fluorescent signal. More preferably, the fluorescent signal is emitted from a fluorescent chemical or a fluorescent protein. Preferred fluorescent chemicals are fluorescein isothiocyanate and rhodamine, and preferred fluorescent proteins are Green Fluorescent Protein, Blue Fluorescent Protein, Cyan Fluorescent Protein, Yellow Fluorescent Protein and Red Fluorescent Protein. Most preferably, the fluorescent signal is modulated by fluorescent resonance energy transfer (FRET). The fluorescent signal is preferably detected by means of a fluorescence activated cell sorter (FACS).

Preferably, the expression of the gene is modulated such that it is 110% or more, 150% or more, 200% or more, 250% or more, 300% or more, 400% or more, 500% or more, or even higher, compared to an unmodulated level. Where the expression of a gene is down-regulated, this is preferably such that the level of expression is 95% or less, 90% or less, 80% or less, 70% or less, 60% or less, 50% or less, 40% or less, 30% or less, 20% or less, 15% or less, or 10% or less than the corresponding un-modulated level.

Furthermore, the expression of more than one gene may be modulated by the expression of one or more heterologous nucleic acid binding polypeptides. Thus, regulation of expression of one gene may have downstream effects, leading to the up-regulation or down-regulation of other genes. Thus, the transgenic animals and methods described here may be used as a basis of identifying genes whose expression is dependent or regulated by the expression of other genes. Thus, in one aspect of the invention, we describe a method of identifying a gene of interest, the method comprising the steps of: (a) providing a transgenic animal comprising a heterologous nucleic acid binding polypeptide which binds to a first target gene and modulates its expression; and (b) detecting the expression of a second gene by the transgenic animal. Such a method may be used as the basis for a differential expression screen. The expression of a gene or genes of interest is compared between a transgenic animal (expressing a nucleic acid binding polypeptide which binds to and modulates the expression of a target gene, typically a different gene from the gene or genes of interest). This is then compared to the expression of the gene or genes in a non-recombinant or non-transgenic or wild-type animal, or an animal of similar genetic background to the transgenic animal, save for the presence or absence of the nucleic acid binding polypeptide encoding sequence.

Furthermore, the transgenic animals and methods described here may be used as a basis of an assay or screen for molecules or compounds or substances which potentially affect or modulate the interaction between a nucleic acid binding polypeptide and its cognate target sequence. Thus, in such a screen, a transgenic animal is provided which carries and expresses a transgene encoding a nucleic acid binding polypeptide. The nucleic acid binding polypeptide is such that it binds to and modulates the expression of a nucleic acid sequence, optionally comprising a sequence encoding a reporter gene. The transgenic animal, and/or the nucleic acid binding polypeptide and/or the nucleic acid binding polypeptide (optionally comprising a reporter sequence) is exposed to a candidate substance or compound (which may be in the form of a library of such compounds), and expression of the nucleic acid assayed. Detection of the reporter gives a measure of the efficiency of modulation of expression by the nucleic acid binding polypeptide. The effectiveness of the candidate compound in modulation this interaction may be detected. Such a compound may be used as a drug to treat or prevent a disease which is characterised by inappropriate gene expression, for example, gene expression which is regulated or modulated by binding of a zinc finger (or other nucleic acid binding polypeptide) to a gene sequence.

In another embodiment, the transgenic animals and methods described here may be used as a basis for genomic studies, i.e., in determing the function of a gene. A transgenic animal is constructed which carries a trangene encoding a nucleic acid binding polypeptide; the nucleic acid binding polypeptide is such that it binds to and modulates the expression, preferably down regulates the expression, of a gene. Observation of a relevant phenotype of the transgenic animal then provides an indication of the function of the gene. Thus, for example, where such an animal exhibits an obese phenotype, for example, it may be concluded that the gene in question whose expression is modulated has a role in regulating obesity. The ability to target any nucleic acid sequence by the use of suitably designed (and/or selected) nucleic acid binding polypeptides such as zinc finger polypeptides, as described in further detail below, enables this application to have wide utility.

In a preferred embodiment, the nucleic acid binding polypeptides comprise zinc finger polyeptides, which are capable of affecting the level of expression of a particular gene within an animal or animal cell. Such an animal may be human or non-human. A suitable gene target may be one that is associated with a particular genetic disease such as Alzheimer's disease, multiple sclerosis, Huntingdon's disease, cancer; one required for infectivity or propagation of viruses such as HIV-1, herpes or hepatitis A, B or C; one which is associated with immune rejection of transplanted tissue (either of host or donor origin); one that is associated with a pathway that provides either useful or unwanted biologically active products; or one which is involved in the production, processing, activation or release of enzymes, cytokines, hormones etc. Suitable gene targets for zinc finger polypeptides and other nucleic acid binding polypeptides include amytoid precursor protein (APP), tau, insulin, CXCR4, CCR5, TNFR, IL-1, IL-2, IL-4, IL-10, IL-13, LDL-R, ApoA, ApoE, K-ras, p53, c-myc haemoglobin, factor VIII, factor IX, CD40, B7, telomerase, β-1,3-galactosyl transferase etc.

We demonstrate up- or down-regulation of the expression of endogenous genes by the use of nucleic acid binding polypeptides, in particular zinc finger polypeptides. Such nucleic acid binding polypeptides may be fused to effector domains such as a transcriptional repressor, a transcriptional activator, a transcriptional insulator, an enzymatic domain or a signalling or targeting sequence or domain, to create chimeric proteins. Suitable effector domains include the KRAB repressor from KOX-1, the engrailed domain (Han et al., EMBO J. 12: 2723-2733 (1993)), or snag repressor domains (Grimes et al., Mol Cell. Biol. 16: 6263-6272 (1996)), VP16 or VP64 activation domains (from herpes simplex virus), or RelA activation domain, CTCF insulator regions, Fok1 endonuclease, DNA methyl transferases, histone deacetylases, the COXIV or F₁ATPase N-terminal presequences (mitochondrial targeting, for review see Rosie, D., The Amphipathic Helix, CRC Press, Ed. Epand, R. M. (1993)), the C-terminal amino acids of human catalase or pig D-amino acid oxidase (peroxisome targeting, Gould et al., J. Cell. Biol. 107: 897 (1988).

The zinc finger polypeptides described here specifically cause the activation or repression of target genes within an animal by binding to specific DNA nucleotide sequences. Such target sequences may be situated in the promoter region of the genes, and a transcriptional effect may be exerted through their effector domains. In the case of gene activation the attached regulatory domain may recruit endogenous factors that promote transcription of the gene, and in the case of gene repression the attached regulatory domain may recruit endogenous factors which help to repress transcription. In addition, by targeting nucleic acid binding polypeptides such as zinc finger polypeptides (which may be engineered) to the promoter and other regions of the target genes, control of gene activity may also be achieved through competition for specific DNA target sequences with endogenous transcription repressor or activator proteins. It will be appreciated that in this case, the nucleic acid binding polypeptides to be used need not comprise any further regulatory domains.

Promoter regions are generally found close to the point of transcription initiation of the said gene and are usually 5′ to the initiation point, although they may be 3′ to the start of gene transcription. However, gene expression can often be controlled from regulatory regions many kilobases from the gene itself, such as from enhancer and locus control regions (LCRs). Sequences within enhancers and within LCRs may therefore also form suitable target sites for the nucleic acid binding polypeptides.

Gene expression may also be controlled at the level of chromatin structure by factors such as the methylation state of cytosine bases and the state of histone acetylation. Hence, the DNA target site of nucleic acid binding polypeptide (such as an engineered zinc finger polypeptide) may be anywhere along the chromosomes of the animal. Preferably, the target site is such that an attached effector domain can exert an effect on the expression of the target gene. Thus, preferred target sites are located in the promoter regions adjacent to the target gene, or immediately 5′ or 3′ to the target gene. Further, target sites may be located within enhancer regions or LCRs. Target sites may also be selected to specifically compete with endogenous transcription factors such as Sp1, c-myc, jun, fos, NFκB or p53 etc.

The expression of many genes may also be achieved by controlling the fate (in particular, the localisation, turnover, degradation, translation, etc) of an associated RNA transcript. RNA molecules often contain sites for RNA-binding proteins, which determine RNA half-life. In response to specific cellular or extracellular signals, such as hormones, chemokines and cytokines, the rate of degradation of a particular RNA molecule may be dramatically altered. For example, the AUF1 protein binds the 3′ untranslated region of cyclin D1 (and other mRNAs) and increases its rate of degradation (Lin et al., Mol. Biol. Cell Biol. 20: 7903-7913 (2000)). Zinc finger polypeptides, whether engineered or not, and other nucleic acid binding polypeptides may also be used to control endogenous gene expression by specifically targeting RNA transcripts to either increase or decrease their half-life within the animal cell.

Target Genes and Nucleotide Sequences

The term “target gene” means a gene or other coding sequence, the expression of which can be affected using compositions and methods described here. A target gene may be an endogenous gene (i.e. one which is normally found in the genome of the animal or animal cell) or a heterologous gene (i.e. one that does not normally exist in the genome of the animal or cell).

Genes that provide suitable targets for the nucleic acid binding polypeptides described here include those involved in diseases such as cardiovascular (low-density lipoprotein receptor, CDH1, ABC1, apolipoproteinA-I, ApoA-II, ApoA-IV, ApoE, lipoprotein lipase, LCAT, SR-BI, CETP etc), inflammatory (IL-1β, IL-1Ra, IL-4, IL-10, IL-13, TNF-α etc), metabolic, infectious (viral, bacteria, fungal, etc), genetic, neurological, rheumatological, dermatological, and musculoskeletal diseases.

Also those genes involved in biochemical pathways that synthesise biologically useful (casein), or unwanted products (lactose) in animal products for human consumption, or those involved in the production of valuable therapeutic (factor VIII, factor IX, IGF-1, insulin, antibodies) or industrial products, and those involved in immune rejection of xenotransplants (porcine alpha-1,3-galactosyltransferase), for the creation of useful transgenic animals (see First, N. L. & Thomson, J. Nat. Biotechnol. 16: 620-621 (1998); Colman, A. Biochem. Soc. Symp. 63: 141-147 (1998); Pennisi, E. Science 279: 646-648 (1998); Whitelaw, B. Nat. Biotechnol. 17: 135-136 (1999); Brink M. F. et al., Theriogenology 53: 139-148 (2000); Smith L. C. et al., Can. Vet. J. 41: 919-924 (2000) and Wolf, E. et al., Exp. Physiol. 85: 615-625 (2000) for reviews).

In particular, we describe nucleic acid binding peptides suitable for the treatment of diseases, syndromes and conditions such as hypertrophic cardiomyopathy, bacterial endocarditis, agyria, amyotrophic lateral sclerosis, tetralogy of fallot, myocarditis, anemia, brachial plexus, neuropathies, hemorrhoids, congenital heart defects, alopecia greata, sickle cell anemia, mitral valve prolapse, autonomic nervous system diseases, alzheimer disease, angina pectoris, rectal diseases, arrhythmogenic right, ventricular dysplasia, acne rosacea, amblyopia, ankylosing spondylitis, atrial fibrillation, cardiac tamponade, acquired immunodeficiency syndrome, amyloidosis, autism, brain neoplasms, central nervous system diseases, colour vision defects, arteriosclerosis, breast diseases, central nervous system infections, colorectal neoplasms, arthritis, behcet's syndrome, breast neoplasms, cerebral palsy, common cold, asthma, bipolar disorder, burns, cervix neoplasms, communication disorders, atherosclerosis, candidiasis, charcot-marie disease, crohn disease, attention deficit disorder, brain injuries, cataract, ulcerative colitis, cumulative trauma disorders, cystic fibrosis, developmental disabilities, eating disorders, erysipelas, fibromyalgia, decubitus ulcer, diabetes, emphysema, escherichia coli infections, folliculitis, deglutition disorders, diabetic foot, encephalitis, oesophageal diseases, food hypersensitivity, dementia, down syndrome, japanese encephalitis, eye neoplasms, dengue, dyslexia, endometriosis, fabry's disease, gastroenteritis, depression, dystonia, chronic fatigue syndrome, gastroesophageal reflux, gaucher's disease, hematologic diseases, hirschsprung disease, hydrocephalus, hyperthyroidism, gingivitis, hemophilia, histiocytosis, hyperhidrosis, hypoglycemia, glaucoma, hepatitis, hiv infections, hyperoxaluria, hypothyroidism, glycogen storage disease, hepatolenticular degeneration, hodgkin disease, hypersensitivity, immunologic deficiency syndromes, hernia, holt-oram syndrome, hypertension, impotence, congestive heart failure, herpes genitalis, huntington's disease, pulmonary hypertension, incontinence, infertility, leukemia, systemic lupus erythematosus, maduromycosis, mental retardation, inflammation, liver neoplasms, lyme disease, malaria, inborn errors of metabolism, inflammatory bowel diseases, long qt syndrome, lymphangiomyomatosis, measles, migraine, influenza, low back pain, lymphedema, melanoma, mouth abnormalities, obstructive lung diseases, lymphoma, meningitis, mucopolysaccharidoses, leprosy, lung neoplasms, macular degeneration, menopause, multiple sclerosis, muscular dystrophy, myofascial pain syndromes, osteoarthritis, pancreatic neoplasms, peptic ulcer, myasthenia gravis, nausea, osteoporosis, panic disorder, myeloma, acoustic neuroma, otitis media, paraplegia, phenylketonuria, myeloproliferative disorders, nystagmus, ovarian neoplasms, parkinson disease, pheochromocytoma, myocardial diseases, opportunistic infections, pain, pars planitis, phobic disorders, myocardial infarction, hereditary optic atrophy, pancreatic diseases, pediculosis, plague, poison ivy dermatitis, prion diseases, reflex sympathetic dystrophy, schizophrenia, shyness, poliomyelitis, prostatic diseases, respiratory tract diseases, scleroderma, sjogren's syndrome, polymyalgia rheumatica, prostatic neoplasms, restless legs, scoliosis, skin diseases, postpoliomyelitis syndrome, psoriasis, retinal diseases, scurvy, skin neoplasms, precancerous conditions, rabies, retinoblastoma, sex disorders, sleep disorders, pregnancy, sarcoidosis, sexually transmitted diseases, spasmodic torticollis, spinal cord injuries, testicular neoplasms, trichotillomania, urinary tract, infections, spinal dystaphism, substance-related disorders, thalassemia, trigeminal neuralgia, urogenital diseases, spinocerebellar degeneration, sudden infant death, thrombosis, tuberculosis, vascular diseases, strabismus, tinnitus, tuberous sclerosis, post-traumatic stress disorders, syringomyelia, tourette syndrome, turner's syndrome, vision disorders, psychological stress, temporomandibular joint dysfunction syndrome, trachoma, urinary incontinence, von willebrand's disease, renal osteodystrophy, bacterial infections, digestive system neoplasms, bone neoplasms, vulvar diseases, ectopic pregnancy, tick-borne diseases, marfan syndrome, aging, williams syndrome, angiogenesis factor, urticaria, sepsis, malabsorption syndromes, wounds and injuries, cerebrovascular accident, multiple chemical sensitivity, dizziness, hydronephrosis, yellow fever, neurogenic arthropathy, hepatocellular carcinoma, pleomorphic adenoma, vater's ampulla, meckel's diverticulum, keratoconus skin, warts, sick building syndrome, urologic diseases, ischemic optic neuropathy, common bile duct calculi, otorhinolaryngologic diseases, superior vena cava syndrome, sinusitis, radius fractures, osteitis deformans, trophoblastic neoplasms, chondrosarcoma, carotid stenosis, varicose veins, creutzfeldt-jakob syndrome, gallbladder diseases, replacement of joint, vitiligo, nose diseases, environmental illness, megacolon, pneumonia, vestibular diseases, cryptococcosis, herpes zoster, fallopian tube neoplasms, infection, arrhythmia, glucose intolerance, neuroendocrine tumors, scabies, alcoholic hepatitis, parasitic diseases, salpingitis, cryptococcal meningitis, intracranial aneurysm, calculi, pigmented nevus, rectal neoplasms, mycoses, hemangioma, colonic neoplasms, hypervitaminosis a, nephrocalcinosis, kidney neoplasms, vitamins, carcinoid tumor, celiac disease, pituitary diseases, brain death, biliary tract diseases, prostatitis, iatrogenic disease, gastrointestinal hemorrhage, adenocarcinoma, toxic megacolon, amputees, seborrheic keratosis, osteomyelitis, barrett esophagus, hemorrhage, stomach neoplasms, chickenpox, cholecystitis, chondroma, bacterial infections and mycoses, parathyroid neoplasms, spermatic cord torsion, adenoma, lichen planus, anal gland neoplasms, lipoma, tinea pedis, alcoholic liver diseases, neurofibromatoses, lymphatic diseases, elder abuse, eczema, diverticulitis, carcinoma, pancreatitis, amebiasis, pyelonephritis, and infectious mononucleosis, etc.

Most commonly, target nucleotide sequences will comprise sequences associated with a target gene that is to be regulated by a nucleic acid binding polypeptide such as a zinc finger polypeptide. The term “target nucleotide sequence” means any nucleic acid sequence to which a nucleic acid binding polypeptide is capable of binding. Examples include DNA sequences within an animal chromosome (but may be an RNA transcript), to which a zinc finger polypeptide (or other nucleic acid binding polypeptide) is capable of binding. A target DNA sequence will generally be associated with a target gene (see above) and the binding of the zinc finger polypeptide or other nucleic acid binding polypeptide to the DNA sequence will generally allow the up- or down-regulation of the associated coding sequence. Target nucleotide sequences include sequences which are naturally associated with target genes, their RNA transcripts, and also other sequences which can be configured with a target gene to allow the up- or down-regulation of such gene. For example, the known binding site of a given nucleic acid binding polypeptide may comprise a target DNA sequence and, when operably linked to a target gene, will allow expression of the target gene to be regulated by the given zinc finger protein. Similarly, the target nucleotide sequence may comprise an RNA sequence within the RNA transcript of the target gene. In this case, binding of the zinc finger polypeptide to the RNA will allow the half-life or targeting of the RNA to be controlled, leading to more or less expression of the associated gene.

With the completion of the human genome project, and the identification of 30-40,000 genes, most of which are completely uncharacterized, many new targets for functional genomic projects have appeared. Zinc finger polypeptides offer a rapid solution to the up- and down-regulation of these genes in transgenic animals (see below). A further advantage of the methods described here is that very short nucleotide sequences associated with target genes are required, against which to design a zinc finger polypeptide or nucleic acid binding polypeptide, rather than the full sequence information required for many other transgenic techniques (see below).

Nucleic Acid Binding Polypeptides

The present invention relates in one aspect to the production and use of nucleic acid binding polypeptides. Such nucleic acid binding polypeptides are preferably engineered. The term “engineered” means that the nucleic acid binding polypeptide, zinc finger polypeptide, polypeptide, protein or fusion protein has been generated or modified in vitro. Typically a zinc finger polypeptide is produced by deliberate mutagenesis, for example the substitution of one or more amino acid residues, either as part of a random mutagenesis procedure or by site-directed mutagenesis, or by selection from a library or libraries of mutated zinc finger polypeptides. Engineered zinc finger polypeptides for use in the methods described here can also be produced de novo using rational design strategies.

The term “polypeptide”, “peptide” and “protein” are used interchangeably to refer to a polymer of amino acid residues, preferably including naturally occurring amino acid residues. Artificial analogues of amino acids may also be used in the nucleic acid binding polypeptides, to impart the proteins with desired properties or for other reasons. Thus, the term “amino acid”, particularly in the context where “any amino acid” is referred to, means any sort of natural or artificial amino acid or amino acid analogue that may be employed in protein construction according to methods known in the art. Moreover, any specific amino acid referred to herein may be replaced by a functional analogue thereof, particularly an artificial functional analogue. Polypeptides may be modified, for example by the addition of carbohydrate residues to form glycoproteins. The nomenclature used herein therefore specifically comprises within its scope functional analogues or mimetics of the defined amino acids.

As used herein, “nucleic acid” includes both RNA and DNA, constructed from natural nucleic acid bases or synthetic bases, or mixtures thereof. Preferably, however, the nucleic acid binding polypeptides comprise DNA binding polypeptides.

Zinc Finger Polypeptides

Particularly preferred examples of nucleic acid binding polypeptides are zinc finger polypeptides. Zinc finger polypeptides typically contain strings of small domains, known as “fingers”, each stabilised by the co-ordination of zinc. Thus, binding of zinc finger polypeptides to target nucleic acid sequences occurs via α-helical zinc metal atom co-ordinated binding motifs known as zinc fingers. Zinc fingers are capable of recognising and binding to a nucleic acid triplet, or an overlapping quadruplet, in a nucleic acid binding sequence. Particularly preferred nucleic acid binding polypeptides comprise zinc finger polypeptides, more preferably zinc finger polypeptides of the Cys2-His2 type.

However, zinc fingers are also known to bind RNA and proteins (Searles, M. A. et al., J. Mol. Biol. 301: 47-60 (2000); Mackay, J. P. & Crossley, M. Trends Biochem. Sci. 23: 1-4).

Preferably, there are 2 or more zinc fingers, for example 2, 3, 4, 5, 6, 7, 8, 9, 10, 11, 12, 13, 14, 15, 16, 17, 18 or more zinc fingers, in each zinc finger polypeptide. Advantageously, the zinc finger polypeptide comprises 3 or more zinc fingers. Furthermore, the number of zinc fingers in a zinc finger polypeptide is preferably a multiple of two.

The DNA binding residue positions of zinc finger polypeptides, as referred to herein, are numbered from the first residue in the α-helix of the finger, ranging from +1 to +9. “−1” refers to the residue in the framework structure immediately preceding the α-helix in a zinc finger polypeptide, for example, a Cys2-His2 zinc finger polypeptide. Residues referred to as “++” are residues present in an adjacent (C-terminal) finger. Where there is no C-terminal adjacent finger, “++” interactions do not operate.

The α-helix of a zinc finger binding protein aligns antiparallel to the nucleic acid strand, such that the primary nucleic acid sequence is arranged 3′ to 5′ in order to correspond with the N-terminal to C-terminal sequence of the zinc finger. Since nucleic acid sequences are conventionally written 5′ to 3′, and amino acid sequences N-terminus to C-terminus, the result is that when a nucleic acid sequence and a zinc finger polypeptide are aligned according to convention, the primary interaction of the zinc finger is with the − strand of the nucleic acid, since it is this strand which is aligned 3′ to 5′. These conventions are followed in the nomenclature used herein. It should be noted, however, that in nature certain fingers, such as finger 4 of the protein GLI, bind to the + strand of the nucleic acid sequence. See Suzuki et al. (1994) Nucl. Acids Rev. 22: 3397-3405; and Pavletich and Pabo, (1993) Science 261: 1701-1707. The present invention encompasses incorporation of such zinc finger polypeptides into DNA binding molecules.

A zinc finger binding motif is a structure well known to those in the art and defined in, for example, Miller et al., (1985) EMBO J. 4:1609-1614; Berg (1988) PNAS (USA) 85:99-102; Lee et al., (1989) Science 245:635-637; see International patent applications WO 96/06166 and WO 96/32475, corresponding to U.S. Ser. No. 08/422,107, incorporated herein by reference.

In general, a preferred zinc finger framework has the structure:
(A) X₀₋₂CX₁₋₅CX₉₋₁₄HX₃₋₆H/C
where X is any amino acid, and the numbers in subscript indicate the possible numbers of residues represented by X.

The above framework may be further refined to include the structure:

$$\begin{matrix}{X}_{0 - 2} C {X}_{1 - 5} C {X}_{2 - 7} & X & X & X & X & X & X & X & H & {X}_{3 - 6} H / C \\ & - 1 & 1 & 2 & 3 & 4 & 5 & 6 & 7 & \end{matrix} \qquad ( {A}^{′} )$$

where X is any amino acid, and the numbers in subscript indicate the possible numbers of residues represented by X.

In a preferred aspect, zinc finger nucleic acid binding motifs may be represented as motifs having the following primary structure:

$$\begin{matrix}{X}^{a} C {X}_{2 - 4} C {X}_{2 - 3} F {X}^{c} & X X X X L X X H X X {X}^{b} H - \\ & linker \\ & - 1 \; 1 \; 2 \; 3 \; 4 \; 5 \; 6 \; 7 \; 8 \; 9\end{matrix} \qquad ( B )$$

wherein X (including X^{a}, X^{b} and X^{c}) is any amino acid. X₂₋₄ and X₂₋₃ refer to the presence of 2 or 4, or 2 or 3, amino acids, respectively.

The Cys and His residues, which together co-ordinate the zinc metal atom, are marked in bold text and are usually invariant, as is the Leu residue at position +4 in the α-helix. Residues X, X^{a}, X^{b}, X^{c} etc are referred to for convenience as “backbone” residues.

Modifications to the standard representation of a zinc finger may occur or be effected without necessarily abolishing zinc finger polypeptide function, by insertion, mutation or deletion of amino acid residues. For example the second His residue may be replaced by Cys (Krizek et al. (1991) J. Am. Chem. Soc. 113: 4518-4523) and that Leu at +4 can in some circumstances be replaced with Arg. The Phe residue before X_{c} may be replaced by any aromatic residue other than Trp. Moreover, experiments have shown that departure from the preferred structure and residue assignments for a zinc finger polypeptide are tolerated and may even prove beneficial in binding to certain nucleic acid sequences. Even taking this into account, however, the general structure involving an α-helix co-ordinated by a zinc atom which contacts four Cys or His residues, is not altered. As used herein, structures (A), (A′) and (B) above are taken as an exemplary structure representing all zinc finger polypeptide structures.

Preferably, X^{a} is F/Y-X or P-F/Y-X. In this context, X is any amino acid. Preferably, in this context X is E, K, T or S. Less preferred but also envisaged are Q, V, A and P. The remaining amino acids remain possible.

Preferably, X₂₋₄ consists of two amino acids rather than four. The first of these amino acids may be any amino acid, but S, E, K, T, P and R are preferred. Advantageously, it is P or R. The second of these amino acids is preferably E, although any amino acid may be used.

Preferably, X^{b} is T or I. Preferably, X^{c} is S or T.

Preferably, X₂₋₃ is G-K-A, G-K-C, G-K-S or G-K-G. However, departures from the preferred residues are possible, for example in the form of M-R-N or M-R.

The linker may comprise a sequence T-G-E/Q-K/R or T-G-E/Q-K/R-P. The linker may comprise a canonical, structured or flexible linker. Structured and flexible linkers (as well as canonical linkers) are described elsewhere in this document, and in our UK application numbers GB 0001582.6, GB0013103.7, GB0013104.5 and our International Patent Application PCT/GB00/00202, all of which are hereby incorporated by reference.

Engineering, Rational and Rule Based Design of Zinc finger Polypeptides

The rules set forth for zinc finger polypeptide design in our European or PCT patent applications having publication numbers WO 98/53057, WO 98/53060, WO 98/53058, WO 98/53059 may be used to design zinc finger proteins for use in the methods described here. These publications describe improved techniques for designing zinc finger polypeptides capable of binding desired nucleic acid sequences. Engineering of zinc finger polypeptides which involves applying rules which specify the choice of amino acid residues based on the identity of residues in a target nucleic acid sequence is referred to here as “rule based” or “rational” design. Such rational design provides a great deal of versatility in zinc finger design.

In combination with selection procedures, such as phage display, set forth for example in WO 96/06166 and described in further detail below, these techniques enable the production of zinc finger polypeptides capable of recognising practically any desired sequence.

The zinc finger polypeptides described here, and for use in the methods described here, may be produced using a method for preparing a zinc finger nucleic acid binding protein capable of binding to a nucleic acid triplet in a target nucleic acid sequence, wherein binding to each base of the triplet by an α-helical zinc finger nucleic acid binding motif in the protein is determined as follows: (a) if the 5′ base in the triplet is G, then position +6 in the α-helix is Arg; or position +6 is Ser or Thr and position ++2 is Asp; (b) if the 5′ base in the triplet is A, then position +6 in the α-helix is Gln and ++2 is not Asp; (c) if the 5′ base in the triplet is T, then position +6 in the α-helix is Ser or Thr and position ++2 is Asp; (d) if the 5′ base in the triplet is C, then position +6 in the α-helix may be any amino acid, provided that position ++2 in the α-helix is not Asp; (e) if the central base in the triplet is G, then position +3 in the α-helix is His; (f) if the central base in the triplet is A, then position +3 in the α-helix is Asn; (g) if the central base in the triplet is T, then position +3 in the α-helix is Ala, Ser or Val; provided that if it is Ala, then one of the residues at −1 or +6 is a small residue; (h) if the central base in the triplet is C, then position +3 in the α-helix is Ser, Asp, Glu, Leu, Thr or Val; (i) if the 3′ base in the triplet is G, then position −1 in the α-helix is Arg; (j) if the 3′ base in the triplet is A, then position −1 in the α-helix is Gln; (k) if the 3′ base in the triplet is T, then position −1 in the α-helix is Asn or Gln; (1) if the 3′ base in the triplet is C, then position −1 in the α-helix is Asp.

Furthermore, a zinc finger nucleic acid binding protein capable of binding to a nucleic acid quadruplet in a target nucleic acid sequence comprising a target nucleotide sequence may be prepared using the following rules. Binding to each base of the quadruplet by an α-helical zinc finger nucleic acid binding motif in the protein is determined as follows: (a) if base 4 in the quadruplet is G, then position +6 in the α-helix is Arg or Lys; (b) if base 4 in the quadruplet is A, then position +6 in the α-helix is Glu, Asn or Val; (c) if base 4 in the quadruplet is T, then position +6 in the α-helix is Ser, Thr, Val or Lys; (d) if base 4 in the quadruplet is C, then position +6 in the α-helix is Ser, Thr, Val, Ala, Glu or Asn; (e) if base 3 in the quadruplet is G, then position +3 in the α-helix is His; (f) if base 3 in the quadruplet is A, then position +3 in the α-helix is Asn; (g) if base 3 in the quadruplet is T, then position +3 in the α-helix is Ala, Ser or Val; provided that if it is Ala, then one of the residues at −1 or +6 is a small residue; (h) if base 3 in the quadruplet is C, then position +3 in the α-helix is Ser, Asp, Glu, Leu, Thr or Val; (i) if base 2 in the quadruplet is G, then position −1 in the α-helix is Arg; (j) if base 2 in the quadruplet is A, then position −1 in the α-helix is Gin; (k) if base 2 in the quadruplet is T, then position −1 in the α-helix is His or Thr; (l) if base 2 in the quadruplet is C, then position −1 in the α-helix is Asp or His; (m) if base 1 in the quadruplet is G, then position +2 is Glu; (n) if base 1 in the quadruplet is A, then position +2 Arg or Gln; (o) if base 1 in the quadruplet is C, then position +2 is Asn, Gln, Arg, His or Lys; (p) if base 1 in the quadruplet is T, then position +2 is Ser or Thr.

The above rules may be further refined, to provide a method for preparing a zinc finger nucleic acid binding protein capable of binding to a nucleic acid quadruplet in a target nucleic acid sequence comprising a target nucleotide sequence, wherein binding to each base of the quadruplet by an α-helical zinc finger nucleic acid binding motif in the protein is determined as follows: (a) if base 4 in the quadruplet is G, then position +6 in the α-helix is Arg; or position +6 is Ser or Thr and position ++2 is Asp; (b) if base 4 in the quadruplet is A, then position +6 in the α-helix is Gln and ++2 is not Asp; (c) if base 4 in the quadruplet is T, then position +6 in the α-helix is Ser or Thr and position ++2 is Asp; (d) if base 4 in the quadruplet is C, then position +6 in the α-helix may be any amino acid, provided that position ++2 in the α-helix is not Asp; (e) if base 3 in the quadruplet is G, then position +3 in the α-helix is His; (f) if base 3 in the quadruplet is A, then position +3 in the α-helix is Asn; (g) if base 3 in the quadruplet is T, then position +3 in the α-helix is Ala, Ser or Val; provided that if it is Ala, then one of the residues at −1 or +6 is a small residue; (h) if base 3 in the quadruplet is C, then position +3 in the α-helix is Ser, Asp, Glu, Leu, Thr or Val; (i) if base 2 in the quadruplet is G, then position −1 in the α-helix is Arg; (j) if base 2 in the quadruplet is A, then position −1 in the α-helix is Gln; (k) if base 2 in the quadruplet is T, then position −1 in the α-helix is Asn or Gln; (1) if base 2 in the quadruplet is C, then position −1 in the α-helix is Asp; (m) if base 1 in the quadruplet is G, then position +2 is Asp; (n) if base 1 in the quadruplet is A, then position +2 is not Asp; (o) if base 1 in the quadruplet is C, then position +2 is not Asp; (p) if base 1 in the quadruplet is T, then position +2 is Ser or Thr.

As set out above, the major binding interactions occur with amino acids −1, +3 and +6. Amino acids +4 and +7 are largely invariant. The remaining amino acids may be essentially any amino acids. Preferably, position +9 is occupied by Arg or Lys. Advantageously, positions +1, +5 and +8 are not hydrophobic amino acids, that is to say are not Phe, Trp or Tyr. Preferably, position ++2 is any amino acid, and preferably serine, save where its nature is dictated by its role as a ++2 amino acid for an N-terminal zinc finger in the same nucleic acid binding molecule.

The foregoing represents sets of rules which permits the design of a zinc finger binding protein specific for any given target DNA sequence. In a most preferred aspect, therefore, the above rules allow the definition of every residue in a zinc finger polypeptide DNA binding motif which will bind specifically to a given target DNA triplet or quadruplet. In order to produce a binding protein having improved binding, moreover, the rules described here may be supplemented by physical or virtual modelling of the protein/DNA interface in order to assist in residue selection.

The code provided by the description above is not entirely rigid; certain choices are provided. For example, positions +1, +5 and +8 may have any amino acid allocation, whilst other positions may have certain options: for example, the present rules provide that, for binding to a central T residue, any one of Ala, Ser or Val may be used at +3. In its broadest sense; therefore, these considerations provide a very large number of proteins which are capable of binding to every defined target DNA triplet.

Preferably, however, the number of possibilities may be significantly reduced. For example, the non-critical residues +1, +5 and +8 may be occupied by the residues Lys, Thr and Gln respectively as a default option. In the case of the other choices, for example, the first-given option may be employed as a default. Thus, the code described here allows the design of a single, defined polypeptide (a “default” polypeptide) which will bind to its target triplet. Zinc finger polypeptides may be based on naturally occurring zinc fingers and consensus zinc fingers.

Accordingly, the zinc finger polypeptides described and for use here can be prepared using a method comprising the steps of: (a) selecting a model zinc finger polypeptide from the group consisting of naturally occurring zinc finger proteins and consensus zinc finger polypeptides; and (b) mutating at least one of positions −1, +3, +6 (and ++2) of the polypeptide.

In general, naturally occurring zinc fingers may be selected from those fingers for which the DNA binding specificity is known. For example, these may be the fingers for which a crystal structure has been resolved: namely Zif268 (Elrod-Erickson et al., (1996) Structure 4:1171-1180), GLI (Pavletich and Pabo, (1993) Science 261:1701-1707), Tramtrack (Fairall et al., (1993) Nature 366:483-487) and YY1 (Houbaviy et al., (1996) PNAS (USA) 93:13577-13582). Preferably, the modified nucleic acid binding polypeptide is derived from Zif 268, GAC, or a Zif-GAC fusion comprising three fingers from Zif linked to three fingers from GAC. By “GAC-clone”, we mean a three-finger variant of Zif268 which is capable of binding the sequence GCGGACGCG, as described in Choo & Klug (1994), Proc. Natl. Acad. Sci. USA, 91, 11163-11167.

Although mutation of the DNA-contacting amino acid residues of the DNA binding domain of zinc finger polypeptides allows selection of peptides which bind to desired target nucleic acids, in a preferred embodiment residues which are outside the DNA-contacting region may be mutated. Mutations in such residues may affect the interaction between zinc finger polypeptides in a zinc finger polypeptide, and thus alter binding site specificity. For instance, Arg at the +10 position of TFIIIA finger 3 makes a base specific contact to guanine (Nolte, R. T. et al., Proc. Natl. Acad. Sci. USA 95: 2938-2943 (1998). Similarly, residues other than those at positions −1, +3, +6 and ++2 may also be utilised for binding RNA molecules.

The naturally occurring zinc finger 2 in Zif268 makes an excellent starting point from which to engineer a zinc finger and is preferred.

Consensus zinc finger structures may be prepared by comparing the sequences of known zinc fingers, irrespective of whether their binding domain is known. Preferably, the consensus structure is selected from the group consisting of the consensus structure P Y K C P E C G K S F S Q K S D L V K H Q R T H T, and the consensus structure P Y K C S E C G K A F S Q K S N L T R H Q R I H T. The consensuses are derived from the consensus provided by Krizek et al., (1991) J. Am. Chem. Soc. 113: 4518-4523 and from Jacobs, (1993) PhD thesis, University of Cambridge, UK. In both cases, canonical, structured or flexible linker sequences, as described below, may be formed on the ends of the consensus for joining two zinc finger domains together.

When the nucleic acid specificity of the model finger selected is known, the mutation of the finger in order to modify its specificity to bind to the target DNA may be directed to residues known to affect binding to bases at which the natural and desired targets differ. Otherwise, mutation of the model fingers should be concentrated upon residues −1, +3, +6 and ++2 as provided for in the foregoing rules.

Selection of Zinc Fingers from Libraries

The rational design described above may be used instead of, or to complement zinc finger production by selection from libraries.

Thus, the zinc finger polypeptides described here are capable of binding to a target DNA sequence comprising a target nucleotide sequence may be produced by a method comprising: a) providing a nucleic acid library encoding a repertoire of zinc finger domains or modules, the nucleic acid members of the library being at least partially randomised at one or more of the positions encoding residues −1, 2, 3 and 6 of the α-helix of the zinc finger modules; b) displaying the library in a selection system and screening it against the target DNA sequence; and c) isolating the nucleic acid members of the library encoding zinc finger modules or domains capable of binding to the target sequence.

The term “library” is used according to its common usage in the art, to denote a collection of polypeptides or, preferably, nucleic acids encoding polypeptides. Methods for the production of libraries encoding randomised members such as polypeptides are known in the art and may be applied here. The members of the library may contain regions of randomisation, such that each library will comprise or encode a repertoire of polypeptides, wherein individual polypeptides differ in sequence from each other. The same principle is present in virtually all-libraries developed for selection, such as by phage display.

Randomisation, as used herein, refers to the variation of the sequence of the polypeptides which comprise the library, such that various amino acids may be present at any given position in different polypeptides. Randomisation may be complete, such that any amino acid may be present at a given position, or partial, such that only certain amino acids are present. Preferably, the randomisation is achieved by mutagenesis at the nucleic acid level, for example by synthesising novel genes encoding mutant proteins and expressing these to obtain a variety of different proteins. Alternatively, existing genes can be themselves mutated, such by site-directed or random mutagenesis, in order to obtain the desired mutant genes.

Zinc finger polypeptides may be designed which specifically bind to nucleic acids incorporating the base U, in preference to the equivalent base T.

A further method for producing a zinc finger polypeptide for use here and capable of binding to a target DNA sequence comprising a target nucleotide sequence comprises: a) providing a nucleic acid library encoding a repertoire of zinc finger polypeptides each possessing more than one zinc finger, the nucleic acid members of the library being at least partially randomised at one or more of the positions encoding residues −1, 2, 3 and 6 of the α-helix in a first zinc finger and at one or more of the positions encoding residues −1, 2, 3 and 6 of the α-helix in a further zinc finger of the zinc finger polypeptides; b) displaying the library in a selection system and screening it against the target DNA sequence; and d) isolating the nucleic acid members of the library encoding zinc finger polypeptides capable of binding to the target sequence.

The library technology described in our International patent application WO 98/53057, incorporated herein by reference in its entirety, may also be employed. WO 98/53057 describes the production of zinc finger polypeptide libraries in which each individual zinc finger polypeptide comprises more than one, for example two or three, zinc fingers; and wherein within each polypeptide partial randomisation occurs in at least two zinc fingers. This allows for the selection of the “overlap” specificity, wherein, within each triplet, the choice of residue for binding to the third nucleotide (read 3′ to 5′ on the + strand) is influenced by the residue present at position +2 on the subsequent zinc finger, which displays cross-strand specificity in binding. The selection of zinc finger polypeptides incorporating cross-strand specificity of adjacent zinc fingers enables the selection of nucleic acid binding proteins more quickly, and/or with a higher degree of specificity than is otherwise possible.

Thus, zinc finger binding motifs designed according to the methods described above may be combined into nucleic acid binding polypeptide molecules having a multiplicity of zinc fingers. Preferably, the proteins have at least two zinc fingers. The presence of at least three zinc fingers is preferred. Nucleic acid binding proteins may be constructed by joining the required fingers end to end, N-terminus to C-terminus, with canonical, flexible or structured linkers, as described elsewhere. Preferably, this is effected by joining together the relevant nucleic acid sequences which encode the zinc fingers to produce a composite nucleic acid coding sequence encoding the entire binding protein. A “leader” peptide may be added to the N-terminal finger. Preferably, the leader peptide is MAEEKP, MAEERP or MAERP. Other polypeptide motifs may be added as desired, for example, nuclear localisation sequences, transcriptional modulator domains such as repressor domains or activation domains, etc.

We therefore describe a method for producing a DNA binding protein for use as described here, wherein the DNA binding protein is constructed by recombinant DNA technology, the method comprising the steps of: preparing a nucleic acid coding sequence encoding a plurality of zinc finger domains or modules defined above, inserting the nucleic acid sequence into a suitable expression vector; and expressing the nucleic acid sequence in a host organism in order to obtain the DNA binding protein.

Flexible and Structured Linkers

The nucleic acid binding polypeptides described here may comprise one or more linker sequences. The linker sequences may comprise one or more flexible linkers, one or more structured linkers, or any combination of flexible and structured linkers. Such linkers are disclosed in our co-pending British Patent Application Numbers 0001582.6, 0013102.9, 0013103.7, 0013104.5 and International Patent Application Number PCT/GB01/00202, which are incorporated by reference.

By “linker sequence” we mean an amino acid sequence that links together two nucleic acid binding modules. For example, in a “wild type” zinc finger protein, the linker sequence is the amino acid sequence lacking secondary structure which lies between the last residue of the α-helix in a zinc finger and the first residue of the β-sheet in the next zinc finger. The linker sequence therefore joins together two zinc fingers. Typically, the last amino acid in a zinc finger is a threonine residue, which caps the α-helix of the zinc finger, while a tyrosine/phenylalanine or another hydrophobic residue is the first amino acid of the following zinc finger. Accordingly, in a “wild type” zinc finger, glycine is the first residue in the linker, and proline is the last residue of the linker. Thus, for example, in the Zif268 construct, the linker sequence is G(E/Q)KP.

A “flexible” linker is an amino acid sequence which does not have a fixed structure (secondary or tertiary structure) in solution. Such a flexible linker is therefore free to adopt a variety of conformations. An example of a flexible linker is the canonical linker sequence GERP/GEKP/GQRP/GQKP. Flexible linkers are also disclosed in WO99/45132 (Kim and Pabo). By “structured linker” we mean an amino acid sequence which adopts a relatively well-defined conformation when in solution. Structured linkers are therefore those which have a particular secondary and/or tertiary structure in solution.

Determination of whether a particular sequence adopts a structure may be done in various ways, for example, by sequence analysis to identify residues likely to participate in protein folding, by comparison to amino acid sequences which are known to adopt certain conformations (e.g., known alpha-helix, beta-sheet or zinc finger sequences), by NMR spectroscopy, by X-ray diffraction of crystallised peptide containing the sequence, etc as known in the art.

The structured linkers preferably do not bind nucleic acid, but where they do, then such binding is not sequence specific. Binding specificity may be assayed for example by gel-shift as described below.

The linker may comprise any amino acid sequence that does not substantially hinder interaction of the nucleic acid binding modules with their respective target subsites. Preferred amino acid residues for flexible linker sequences include, but are not limited to, glycine, alanine, serine, threonine proline, lysine, arginine, glutamine and glutamic acid.

The linker sequences between the nucleic acid binding domains preferably comprise five or more amino acid residues. The flexible linker sequences preferably consist of 5 or more residues, preferably, 5, 6, 7, 8, 9, 10, 11, 12, 13, 14, 15, 16, 17, 18, 19 or 20 or more residues. In a highly preferred embodiment, the flexible linker sequences consist of 5, 7 or 10 residues.

Once the length of the amino acid sequence has been selected, the sequence of the linker may be selected, for example by phage display technology (see for example U.S. Pat. No. 5,260,203) or using naturally occurring or synthetic linker sequences as a scaffold (for example, GQKP and GEKP, see Liu et al., 1997, Proc. Natl. Acad. Sci. USA 94, 5525-5530 and Whitlow et al., 1991, Methods: A Companion to Methods in Enzymology 2: 97-105). The linker sequence may be provided by insertion of one or more amino acid residues into an existing linker sequence of the nucleic acid binding polypeptide. The inserted residues may include glycine and/or serine residues. Preferably, the existing linker sequence is a canonical linker sequence selected from GEKP, GERP, GQKP and GQRP. More preferably, each of the linker sequences comprises a sequence selected from GGEKP, GGQKP, GSERP, GGSGEKP, GGSGQKP, GGGGSERP, GGSGGSGEKP, and GGSGGSGQKP.

Structured linker sequences are typically of a size sufficient to confer secondary or tertiary structure to the linker; such linkers may be up to 30, 40 or 50 amino acids long. In a preferred embodiment, the structured linkers are derived from known zinc fingers which do not bind nucleic acid, or are not capable of binding nucleic acid specifically. An example of a structured linker of the first type is TFIIIA finger IV; the crystal structure of TFIIIA has been solved, and this shows that finger IV does not contact the nucleic acid (Nolte et al., 1998, Proc. Natl. Acad. Sci. USA 95, 2938-2943.). An example of the latter type of structured linker is a zinc finger which has been mutagenised at one or more of its base contacting residues to abolish its specific nucleic acid binding capability. Thus, for example, Zif268 finger 2 which has residues −1, 2, 3 and 6 of the recognition helix mutated to serines so that it no longer specifically binds DNA may be used as a structured linker to link two nucleic acid binding domains.

The use of structured or rigid linkers to jump the minor groove of DNA is likely to be especially beneficial in (i) linking zinc fingers that bind to widely separated (>3 bp) DNA sequences, and (ii) also in minimising the loss of binding energy due to entropic factors.

Typically, the linkers are made using recombinant nucleic acids encoding the linker and the nucleic acid binding modules, which are fused via the linker amino acid sequence. The linkers may also be made using peptide synthesis and then linked to the nucleic acid binding modules. Methods of manipulating nucleic acids and peptide synthesis methods are known in the art (see, for example, Maniatis, et al., 1991. Molecular Cloning: A Laboratory Manual. Cold Spring Harbor, N.Y., Cold Spring Harbor Laboratory Press).

Zinc finger polypeptides may also be linked non-covalently. Non-covalent dimerisation domains such as leucine zippers, and coiled coils are preferable for this purpose (O'Shea, Science, 254: 539 (1991); Klemm et al., Ann. Rev. Immunol. 16: 569-592 (1998); Ho, et al., Nature, 382: 822-826 (1996); Pomeranz, et al., Biochem. 37: 965 (1998).

Chimeric Nucleic Acid Binding Polypeptides

In a preferred embodiment, the nucleic acid binding polypeptides described here comprise chimeric nucleic acid binding polypeptides.

A chimeric nucleic acid binding polypeptide comprises a nucleotide binding domain (comprising a number of nucleic acid binding polypeptide modules or fingers) designed to bind specifically to a nucleotide sequence, together with one or more further biological effector domains. The term “biological effector domain” should be taken to mean any polypeptide that has a biological function. Included are enzymes, receptors, regulatory domains, activation or repression domains, binding sequences, dimerisation, trimerisation or multimerisation sequences, sequences involved in protein transport, localisation sequences such as subcellular localisation sequences, nuclear localisation, protein targeting or signal sequences. Furthermore, biological effector domains may comprise polypeptides involved in chromatin remodelling, chromatin condensation or decondensation, DNA replication, transcription, translation, protein synthesis, etc. Fragments of such polypeptides comprising the relevant activity are also included in this definition. Preferred biological effector domains include transcriptional modulation domains such as transcriptional activators and transcriptional repressors.

The effector domain(s) may be covalently or non-covalently attached to the nucleotide-binding domain.

Chimeric nucleic acid binding polypeptides preferably comprise transcription factor activity, for example, a transcriptional modulation activity such as transcriptional activator or transcriptional repressor activity. For example, a zinc finger chimeric polypeptide may comprise a nucleotide binding domain designed to bind specifically to a particular nucleotide sequence, and one or more further biological effector domains, preferably a transcriptional activator or repressor domain, as described in further detail below. The zinc finger chimeric polypeptide may comprise one or more zinc fingers or zinc finger binding modules.

Preferably, in the case of a chimeric polypeptide comprising transcriptional modulation activity, a nuclear localization domain is attached to the DNA binding domain to direct the chimeric polypeptide to the nucleus.

Generally, the chimeric nucleic acid binding polypeptide such as a chimeric zinc finger polypeptide may also include an effector domain to regulate gene expression. The effector domain may be directly derived from a basal or regulated transcription factor such as a transactivator, repressor, insulator or silencer (Choo & Klug (1995) Curr. Opin. Biotech. 6: 431-436; Choo & Klug (1997); Rebar & Pabo (1994) Science 263: 671-673; Jamieson et al. (1994) Biochem. 33: 5689-5695; Goodrich et al., Cell 84: 825-830 (1996); CTCF (Vostrov, A. A. & Quitschke, W. W. J. Biol. Chem. 272: 33353-33359 (1997)). Other useful domains may be derived from membrane receptors such as nuclear hormone receptors (Kumar, R & Thompson, E. B. Steroids 64: 310-319 (1999)), and their co-activators and co-repressors (Ugai, H. et al., J. Mol. Med. 77: 481-494 (1999)).

The chimeric nucleic acid binding polypeptide such as a chimeric zinc finger polypeptide may also preferably include other domains that may be advantageous within the context of the control of gene expression. These domains may include protein-modifying domains such as histone acetyltransferases, kinases and phosphatases, which can silence or activate genes by modifying DNA structure or the proteins that associate with nucleic acids (Wolffe, Science 272: 371-372 (1996); Taunton et al., Science 272: 408-411 (1996); Hassig et al., Proc. Natl. Acad. Sci. USA 95: 3519-3524 (1998); Wang, Trends Biochem. Sci. 19: 373-376 (1994); and Schonthal & Semin, Cancer Biol. 6: 239-248 (1995)). Additional useful effector domains include those that modify or rearrange nucleic acid molecules such as methyltransferases, endonucleases, ligases, recombinases etc. (Wood, Ann. Rev. Biochem. 65: 135-167 (1996); Sadowski, FASEB J. 7: 760-767 (1993); Cheng, Curr. Opin. Struct. Biol. 5: 4-10 (1995)) (Wu et al. (1995) Proc. Natl. Acad. Sci. USA 92:344-348; Nahon & Raveh (1998); Smith et al. (1999); and Carroll et al. (1999)). It will be appreciated that the biological effector domain portion of the chimeric polypeptide may itself also comprise such activities, without the need for further domains.

In one embodiment, the VP64 domain from herpes simplex virus (HSV) is used to activate gene expression (Seipel et al., EMBO J. 11: 4961-4968 (1996). Other preferred transactivator domains include the HSV VP16 domain (Hagmann et al., J. Virol. 71: 5952-5962 (1997), transactivation domain 1 and/or domain 2 of the p65 subunit of nuclear factor-κB (NF-κB, Schmitz, M. L. et al., J. Biol. Chem. 270: 15576-15584 (1995)). Other transcription factors are reviewed in, for example, Lekstrom-Himes J. & Xanthopoulos K. G. (C/EBP family, J. Biol. Chem. 273: 28545-28548 (1998)), Bieker, J. J. et al., (globin gene transcription factors, Ann. N.Y. Acad. Sci. 850: 64-69 (1998), and Parker, M. G. (oestrogen receptors, Biochem. Soc. Symp. 63: 45-50 (1998)).

Use of a transactivation domain from the estrogen receptor is disclosed in Metivier, R., Petit, F G., Valotaire, Y. & Pakdel, F. (2000) Mol. Endocrinol. 14: 1849-1871. Furthermore, activation domains from the globin transcription factors EKLF (Pandya, K. Donze, D.& Townes T. (2001) J. Biol. Chem. 276: 8239-8243) may also be used, as well as a transactivation domain from FKLF (Asano, H. Li, XS.& Stamatoyannopoulos, G. (1999) Mol. Cell. Biol. 19: 3571-3579). C/EPB transactivation domains may also be employed in the methods described here. The C/EBP epsilon activation domain is disclosed in Verbeek, W., Gombart, A F, Chumakov, A M, Muller, C, Friedman, A D, & Koeffler, H P (1999) Blood 15: 3327-10-3337. Kowenz-Leutz, E. & Leutz, A. (1999) Mol. Cell. 4: 735-743 discloses the use of the C/EBP tao activation domain, while the C/EBP alpha transactivation domain is disclosed in Tao, H., & Umek, R M. (1999) DNA Cell Biol. 18: 75-84.

It is known that zinc finger proteins may be fused to transcriptional repression domains such as the Kruppel-associated box (KRAB) domain to form powerful repressors. These fusions are known to repress expression of a reporter gene even when bound to sites a few kilobase pairs upstream from the promoter of the gene (Margolin et al., 1994, Proc. Natl. Acad. Sci. USA 91: 4509-4513). In one preferred embodiment, the KRAB repressor domain from the human KOX-1 protein is used to repress gene activity (Moosmann et al., Biol. Chem. 378: 669-677 (1997); Thiesen et al., New Biologist 2: 363-374 (1990)). Other preferred transcriptional repressor domains are known in the art and include, for example, the engrailed domain (Han et al., EMBO J. 12: 2723-2733 (1993)) and the snag domain (Grimes et al., Mol Cell. Biol. 16: 6263-6272 (1996)). These can be used alone or in combination to down-regulate gene expression in animals.

Biological effector domains may be covalently or non-covalently linked to the nucleotide-binding domain. In a preferred embodiment the covalent linker comprises a amino acid sequence which may be flexible; polypeptides according to this embodiment preferably comprise fusion proteins comprising the nucleic acid binding portion of the chimeric polypeptide fused with an amino acid linker to the biological effector domain portion. Alternatively, the covalent linker may comprise a synthetic, non-amino acid based, chemical linker, for example, polyethylene glycol. Synthetic linkers are commercially available, and methods of chemical conjugation are known in the art. The covalent linkers may comprise flexible or structured linkers, as described in detail above.

Non-covalent linkages between the nucleic acid binding portion and the effector portion may for example be formed using leucine zipper/coiled coil domains, or other naturally occurring or synthetic dimerisation domains (see e.g. Luscher, B. & Larsson, L. G. Oncogene 18:2955-2966 (1999) and Gouldson, P. R. et al., Neuropsychopharmacology 23: S60-S77 (2000)).

The expression of nucleic acid binding polypeptides (for example, zinc finger polypeptides) may be controlled by tissue specific promoter sequences such as the lck promoter (thymocytes, Gu, H. et al., Science 265: 103-106 (1994)); the human CD2 promoter (T-cells and thymocytes, Zhumabekov, T. et al., J. Immunological Methods 185: 133-140 (1995)); the alpha A-crystallin promoter (eye lens, Lakso, M. et al., Proc. Natl. Acad. Sci. 89: 6232-6236 (1992)); the alpha-calcium-calmodulin-dependent kinase II promoter (hippocampus and neocortex, Tsien, J. et al., Cell 87: 1327-1338 (1996)); the whey acidic protein promoter (mammary gland, Wagner, K.-U. et al., Nucleic Acids Res. 25: 4323-4330 (1997)); the aP2 enhancer/promoter (adipose tissue, Barlow C. et al., Nucleic Acids Res. 25: 2543-2545 (1997)); the aquaporin-2 promoter (renal collecting duct, Nelson R. et al., Am. J. Physiol. 275: C216-C226 (1998)); and the mouse myogenin promoter (skeletal muscle, Grieshammer, U. et al., Dev. Biol. 197: 234-247 (1998)). The expression of such polypeptides may also be controlled by inducible systems, in particular, controlled by small molecule induction such as the tetracycline-controlled systems (tet-on and tet-off), the RU-486 or tamoxifen hormone analogue systems, or the radiation-inducible early growth response gene-1 (EGR1) promoter. These promoter constructs and inducible systems have the benefit of being able to give organ specific and inducible expression of target genes for use in applications such as gene therapy and transgenic animals.

Vectors

The nucleic acid encoding the nucleic acid binding polypeptide such as a zinc finger polypeptide may be incorporated into intermediate vectors and transformed into prokaryotic or eukaryotic cells for expression or DNA amplification.

As used herein, vector (or plasmid) preferably refers to discrete elements that are used to introduce heterologous nucleic acid into cells for either expression or replication thereof. The term “heterologous to the cell” means that the sequence does not naturally exist in the genome of the cell but has been introduced into the cell. The term “introduced into” means that a procedure is performed on an animal, an animal organ, or an animal cell such that the gene encoding the nucleic acid binding polypeptide (for example, a zinc finger polypeptide) is then present in the cell or cells. A heterologous sequence may include a modified sequence introduced at any chromosomal site, or which is not integrated into a chromosome, or which is introduced by homologous recombination such that it is present in the genome in the same position as the native allele. Selection and use of such vectors are well within the skill of the person of ordinary skill in the art. Many vectors are available, and selection of an appropriate vector will depend on the intended use of the vector, i.e. whether it is to be used for DNA amplification or for nucleic acid expression, the size of the DNA to be inserted into the vector, and the host cell to be transformed with the vector, etc. Another consideration is whether the vector is to remain episomal or integrate into the host genome. Suitable vectors may be of bacterial, viral, insect or mammalian origin. Intermediate vectors for storage or manipulation of the nucleic acid encoding the nucleic acid binding polypeptide, or for expression and purification of the polypeptide are typically of prokaryotic origin. Most expression vectors are shuttle vectors, i.e. they are capable of replication in at least one class of organisms but can be transfected into another class of organisms for expression. For example, a vector is cloned in E. coli and then the same vector is transfected into yeast or mammalian cells even though it is not capable of replicating independently of the host cell chromosome. DNA may also be replicated by insertion into the host genome. The nucleic acid binding polypeptides such as zinc finger polypeptides described here are preferably inserted into a vector suitable for expression in mammalian cells.

Prokaryote, yeast and higher eukaryote cells may be used for replicating DNA and producing the nucleic acid binding protein. Suitable prokaryotes include eubacteria, such as Gram-negative or Gram-positive organisms, such as E. coli, e.g. E. coli K-12 strains, DH5a and HB101, or Bacilli. Further hosts suitable for the vectors include eukaryotic microbes such as filamentous fungi or yeast, e.g. Saccharomyces cerevisiae. Higher eukaryotic cells include insect and vertebrate cells, particularly mammalian cells including human cells or nucleated cells from other multicellular organisms. In recent years propagation of vertebrate cells in culture (tissue culture) has become a routine procedure. Examples of useful mammalian host cell lines are epithelial or fibroblastic cell lines such as Chinese hamster ovary (CHO) cells, NIH 3T3 cells, HeLa cells or 293T cells. The host cells referred to in this disclosure comprise cells in in vitro culture as well as cells that are within a host animal.

Each vector contains various components depending on its function (amplification of DNA or expression of DNA) and the host cell for which it is compatible. The vector components generally include, but are not limited to, one or more of the following: an origin of replication, one or more selectable marker genes, a promoter, an enhancer element, a transcription termination sequence and a signal sequence.

Both expression and cloning vectors generally contain nucleic acid sequence that enable the vector to replicate in one or more selected host cells. Typically in cloning vectors, this sequence is one that enables the vector to replicate independently of the host chromosomal DNA, and includes origins of replication or autonomously replicating sequences. Such sequences are well known for a variety of bacteria, yeast and viruses. The origin of replication from the plasmid pBR322 is suitable for most Gram-negative bacteria, the 2μ plasmid origin is suitable for yeast, and various viral origins (e.g. SV 40, polyoma, adenovirus) are useful for cloning vectors in mammalian cells. Generally, the origin of replication component is not needed for mammalian expression vectors unless these are used in mammalian cells competent for high level DNA replication, such as COS cells.

Advantageously, an expression and cloning vector contains a selection gene also referred to as selectable marker. This gene encodes a protein necessary for the survival or growth of transformed host cells grown in a selective culture medium. Host cells not transformed with the vector containing the selection gene will not survive in the culture medium. Typical selection genes encode proteins that confer resistance to antibiotics and other toxins, e.g. ampicillin, neomycin, methotrexate or tetracycline, complement auxotrophic deficiencies, or supply critical nutrients not available from complex media.

Since the replication of vectors is conveniently done in E. coli, an E. coli genetic marker and an E. coli origin of replication are advantageously included. These can be obtained from E. coli plasmids, such as pBR322, Bluescript© vector or a pUC plasmid, e.g. pUC18 or pUC19, which contain both E. coli replication origin and E. coli genetic marker conferring resistance to antibiotics, such as ampicillin and tetracycline. Vectors such as these are commercially available.

Suitable selectable markers for mammalian cells are those that enable the identification of cells competent to take up nucleic acid binding protein nucleic acid, such as dihydrofolate reductase (DHFR, methotrexate resistance), thymidine kinase, or genes conferring resistance to G418 or hygromycin. The mammalian cell transformants are placed under selection pressure which only those transformants which have taken up and are expressing the marker are uniquely adapted to survive. In the case of a DHFR or glutamine synthase (GS) marker, selection pressure can be imposed by culturing the transformants under conditions in which the pressure is progressively increased, thereby leading to amplification (at its chromosomal integration site) of both the selection gene and the linked DNA that encodes the nucleic acid binding protein. Amplification is the process by which genes in greater demand (such as a protein that is critical for growth), together with closely associated genes (such as a zinc finger polypeptide), are reiterated in tandem within the chromosomes of recombinant cells. Increased quantities of desired protein are usually synthesised from this amplified DNA.

Expression and cloning vectors usually contain control sequences that are recognised by the host organism and are operably linked to the nucleic acid encoding a nucleic acid binding polypeptide. The term “control sequences” is intended to include, at a minimum, components whose presence can influence expression, and can also include additional components whose presence is advantageous, for example, leader sequences and fusion partner sequences. The term “operably linked” means that the components described are in a relationship permitting them to function in their intended manner. Typical control sequences include promoters, enhancers and other expression regulation signals such as terminators. Such a promoter may be inducible or constitutive. A regulatory sequence operably linked to a coding sequence is ligated in such a way that expression of the coding sequence is achieved under conditions compatible with the control sequences.

The term promoter is well known in the art and encompasses nucleic acid regions ranging in size and complexity from minimal promoters to promoters including upstream elements and enhancers. Suitable promoters for use in prokaryotic and eukaryotic cells are well known in the art, and described in for example, Current Protocols in Molecular Biology (Ausubel et al., eds., 1994) and Molecular Cloning. A Laboratory Manual (Sambrook et al., 2^{nd} ed. 1989).

Promoters suitable for use with prokaryotic hosts include, for example, the β-lactamase and lactose promoter systems, alkaline phosphatase, the tryptophan (Trp) promoter system and hybrid promoters such as the tac promoter. Their nucleotide sequences have been published, thereby enabling the skilled worker operably to ligate them to DNA encoding nucleic acid binding protein, using linkers or adapters to supply any required restriction sites. Promoters for use in bacterial systems will also generally contain a Shine-Delgarno sequence operably linked to the DNA encoding the nucleic acid binding protein.

Preferred expression vectors are bacterial expression vectors, which comprise a promoter of a bacteriophage such as phagex or T7 which is capable of functioning in the bacteria. In one of the most widely used expression systems, the nucleic acid encoding the fusion protein may be transcribed from the vector by T7 RNA polymerase (Studier et al, Methods in Enzymol. 185: 60-89, 1990). In the E. coli BL21(DE3) host strain, used in conjunction with pET vectors, the T7 RNA polymerase is produced from the λ-lysogen DE3 in the host bacterium, and its expression is under the control of the IPTG inducible lac UV5 promoter. This system has been employed successfully for over-production of many proteins. Alternatively, the polymerase gene may be introduced on a lambda phage by infection with an int-phage such as the CE6 phage, which is commercially available (Novagen, Madison, USA). Other vectors include vectors containing the lambda PL promoter such as PLEX (Invitrogen, NL), vectors containing the trc promoters such as pTrcHisXpress™ (Invitrogen), or pTrc99 (Pharmacia Biotech, SE), or vectors containing the tac promoter such as pKK223-3 (Pharmacia Biotech), or PMAL (New England Biolabs, MA, USA). A suitable vector for expression of proteins in mammalian cells is the CMV enhancer-based vector such as pEVRF (Matthias, et al., (1989) Nucleic Acids Res. 17, 6418).

Suitable promoting sequences for use with yeast hosts may be regulated or constitutive and are preferably derived from a highly expressed yeast gene, especially a Saccharomyces cerevisiae gene. Thus, the promoter of the TRP1 gene, the ADHI or ADHII gene, the acid phosphatase (PH05) gene, a promoter of the yeast mating pheromone genes coding for the a- or α-factor or a promoter derived from a gene encoding a glycolytic enzyme such as the promoter of the enolase, glyceraldehyde-3-phosphate dehydrogenase (GAP), 3-phosphoglycerate kinase (PGK), hexokinase, pyruvate decarboxylase, phosphofructokinase, glucose-6-phosphate isomerase, 3-phosphoglycerate mutase, pyruvate kinase, triose phosphate isomerase, phosphoglucose isomerase or glucokinase genes, or a promoter from the TATA binding protein (TBP) gene can be used. Furthermore, it is possible to use hybrid promoters comprising upstream activation sequences (UAS) of one yeast gene and downstream promoter elements including a functional TATA box of another yeast gene, for example a hybrid promoter including the UAS(s) of the yeast PH05 gene and downstream promoter elements including a functional TATA box of the yeast GAP gene (PH05-GAP hybrid promoter). A suitable constitutive PHO5 promoter is, for example, a shortened acid phosphatase PH05 promoter devoid of the upstream regulatory elements (UAS) such as the PH05 (−173) promoter element starting at nucleotide −173 and ending at nucleotide −9 of the PH05 gene.

The promoter is typically selected from promoters which are found in animal cells, although prokaryotic promoters and promoters functional in other eukaryotic cells can be used. Typically, the promoter is derived from viral or animal gene sequences, may be constitutive or inducible, and may be strong or weak.

Commonly used viral promoters are derived from viruses such as polyoma virus, adenovirus, fowlpox virus, bovine papilloma virus, avian sarcoma virus, cytomegalovirus (CMV), a retrovirus and simian virus 40 (SV40). An example of a relatively weak viral promoter is HSV TK, from herpes simplex virus.

Mammalian derived promoters may be heterologous to the animal in which nucleic acid binding polypeptide (such as zinc finger polypeptide) expression is to occur, or may be host sequences. In some applications it is preferable to use a promoter that is active in all cell types, however it is often preferable to use promoter sequences that are active in specific cell types only.

The actin promoter and the strong ribosomal protein promoter are examples of promoter sequences that are active in all cell types. In contrast, by using promoters that are specific for certain cell or tissue types, the gene encoding the nucleic acid binding polypeptide can be expressed only in the required cell or tissue types. This may be of extreme importance for applications such as gene therapy, and for the production of viable transgenic animals. Such promoters are known in the art and include the lck promoter (thymocytes, Gu, H. et al., Science 265: 103-106 (1994)), the human CD2 promoter (T-cells and thymocytes, Zhumabekov, T. et al., J. Immunological Methods 185: 133-140 (1995)); the alpha A-crystallin promoter (eye lens, Lakso, M. et al. Proc. Natl. Acad. Sci. 89: 6232-6236 (1992)), the alpha-calcium-calmodulin-dependent kinase II promoter (hippocampus and neocortex, Tsien, J. et al., Cell 87: 1327-1338 (1996)), the whey acidic protein promoter (mammary gland, Wagner, K.-U. et al., Nucleic Acids Res. 25: 4323-4330 (1997)), the aP2 enhancer/promoter (adipose tissue, Barlow C. et al., Nucleic Acids Res. 25: 2543-2545 (1997)), the aquaporin-2 promoter (renal collecting duct, Nelson R. et al., Am. J. Physiol. 275: C216-C226 (1998)), the mouse myogenin promoter (skeletal muscle, Grieshammer, U. et al., Dev. Biol. 197: 234-247 (1998)), retinoblastoma gene promoter (nervous system, Jiang, Z. et al., J. Biol. Chem. 276: 593-600 (2001)).

The expression of nucleic acid binding polypeptides such as zinc finger polypeptides can also be controlled by small molecule induction or other inducible systems such as the tetracycline inducible systems (tet-on and tet-off), the RU486 or tamoxifen hormone analogue systems, or the radiation-inducible early growth response gene-1 (EGR1) promoter, all of which are commercially available. By using such inducible promoter systems, transgenic lines can be established which carry a zinc finger chimeric polypeptide but express it only after addition of an inducer molecule. Thus the genes encoding the zinc finger polypeptides or other nucleic acid binding polypeptides can be expressed (or not expressed) in response to the small molecule, which can be easily administered. These systems may also allow the time and amount of polypeptide expression to be regulated.

Expression vectors typically contain expression cassettes that carry all the additional elements required for efficient expression of the nucleic acid in the host cell. Additional elements are enhancer sequences, polyadenylation and transcriptional termination signals, ribosome binding sites, and translational termination sequences.

Transcription of DNA by higher eukaryotes may be increased by inserting an enhancer sequence into the vector. Enhancers are relatively orientation and position independent. Many enhancer sequences are known from mammalian genes (e.g. elastase and globin). However, typically one will employ an enhancer from a eukaryotic cell virus. Examples include the SV40 enhancer on the late side of the replication origin (bp 100-270) and the CMV early promoter enhancer. The enhancer may be spliced into the vector at a position 5′ or 3′ to the gene encoding the zinc finger polypeptide or nucleic acid binding polypeptide, but is preferably located at a site 5′ from the promoter.

It has also been shown that the expression of a heterologous gene in an animal cell may be enhanced by retaining intron sequences (as opposed to using a cDNA clone). For example, intron 1 of the human CD2 gene has been shown to enhance the level of expression of CD2 in human cells (Festenstein, R. et al. 1996 Science 271: 1123).

Advantageously, a eukaryotic expression vector encoding a nucleic acid binding protein may comprise a locus control region (LCR). LCRs are capable of directing high-level integration site-independent expression of transgenes integrated into host cell chromatin. This is particularly important where the gene encoding the zinc finger polypeptide or the nucleic acid binding polypeptide is to be expressed over extended periods of time, for applications such as transgenic animals and gene therapy, as gene silencing of integrated heterologous DNA—especially of viral origin—is known to occur (Palmer, T. D. et al., Proc. Natl. Acad. Sci. USA 88: 1330-1334 (1991); Harpers, K. et al., Nature 293: 540-542 (1981); Jahner, D. et al., Nature 298: 623-628 (1992); and Chen, W. Y. et al., Proc. Natl. Acad. Sci. USA 94: 5798-5803 (1997)). Typical LCRs are exemplified by the human β-globin cluster, and the HS-40 regulatory region from the α-globin locus.

Eukaryotic vectors may also contain sequences necessary for the termination of transcription and for stabilising the mRNA transcript. Such sequences are commonly available from the 5′ and 3′ untranslated regions of eukaryotic or viral DNAs, and are known in the art. These regions contain nucleotide segments transcribed as polyadenylated fragments in the untranslated portion of the mRNA encoding the relevant polypeptide. An appropriate terminator of transcription is fused downstream of the gene encoding the selected nucleic acid binding polypeptide such as a zinc finger protein. Any of a number of known transcriptional terminator, RNA polymerase pause sites and polyadenylation enhancing sequences can be used at the 3′ end of the nucleic acid encoding for example a zinc finger polypeptide (see, for example, Richardson, J. P. Crit. Rev. Biochem. Mol. Biol. 28:1-30 (1993); Yonaha M. & Proudfoot, N. J. EMBO J. 19: 3770-3777 (2000); Ashfield, R. et al., EMBO J. 10: 4197-4207 (1991); Hirose, Y. & Manley, J. L. Nature 395: 93-96 (1998)).

The nucleic acid binding polypeptides are generally targeted to the cell nucleus so that they are able to interact with host cell DNA and bind to the appropriate DNA target in the nucleus and regulate transcription. To effect this, a nuclear localization sequence (NLS) is incorporated in frame with the expressible nucleic acid binding polypeptide (e.g., zinc finger polypeptide) gene construct. The NLS can be fused either 5′ or 3′ to the sequence encoding the binding protein, but preferably it is fused to the C-terminus of the chimeric polypeptide.

The NLS of the wild-type Simian Virus 40 Large T-Antigen (Kalderon et al. (1984) Cell 37: 801-813; and Markland et al. (1987) Mol. Cell. Biol. 7: 4255-4265) is an appropriate NLS and provides an effective nuclear localization mechanism in animals. However, several alternative NLSs are known in the art and can be used instead of the SV46 NLS sequence. These include the NLSs of TGA-1A and TGA-1B.

Nucleic acid binding molecules may comprise tag sequences to facilitate studies and/or preparation of such molecules. Tag sequences may include flag-tag, myc-tag, HA-tag, 6his-tag or any other suitable tag known in the art.

Construction of vectors according employs conventional ligation techniques. Isolated plasmids or DNA fragments are cleaved, tailored, and religated in the form desired to generate the plasmids required. If desired, analysis to confirm correct sequences in the constructed plasmids is performed in a known fashion. Suitable methods for constructing expression vectors, preparing in vitro transcripts, introducing DNA into host cells, and performing analyses for assessing nucleic acid binding protein expression and function are known to those skilled in the art. Gene presence, amplification and/or expression may be measured in a sample directly, for example, by conventional Southern blotting, Northern blotting to quantify the transcription of mRNA, dot blotting (DNA or RNA analysis), or in situ hybridisation, using an appropriately labelled probe which may be based on a sequence provided herein. Those skilled in the art will readily envisage how these methods may be modified, if desired.

Transformation and Transfection

DNA can be stably incorporated into cells or can be transiently expressed using methods known in the art and described below. Stably transfected cells can be prepared by transfecting cells with an expression vector containing a selectable marker gene, and growing the transfected cells under conditions selective for cells expressing the marker gene. To prepare transient transfectants, cells are transfected with a reporter gene to monitor transfection efficiency.

There are many well-known methods of introducing foreign nucleic acids into host cells, which include electroporation, calcium phosphate co-precipitation, particle bombardment, microinjection, naked DNA, liposomes, lipofection, and viral infection etc (see, e.g. Sambrook et al. (1989) Molecular Cloning: A Laboratory Manual, Second Edition, Cold Spring Harbor Laboratory Press, and Mountain, A. Trends Biotechnol. 18: 119-128 (2000) for a review). Any of the above methods can be used, as long as it is compatible with the host cell. Linear nucleic acid molecules have been found to be more efficiently incorporated into mammalian genomes than circular plasmids. Additionally, nucleic acid molecules may be delivered in vivo, to specific target tissues, or ex vivo, to individual cells. Viral based gene transfer is often favoured for introducing nucleic acids into mammalian cells and specific target tissues, and several viral delivery approaches are in clinical trials for gene therapy applications. However, non-viral methods are attractive due to their greater safety for the purpose of gene transfer to humans.

The preferred methods of particle bombardment use bolistics made from gold (or tungsten). Compared with other transformation procedures, particle bombardment requires a low amount of nucleic acid and a smaller number of cells, making the procedure generally more efficient (Heiser, W. C. Anal. Biochem. 217: 185-196 (1994); Klein, T. M. & Fitzpatrick-McElligott, S. Curr. Opin. Biotechnol. 4: 583-590 (1993)). The procedure is particularly suited for difficult-to-transform organisms and for introducing DNA into organelles, such as mitochondria and chloroplasts. Although, generally used for ex vivo applications, the procedure is also suitable for in vivo transformation of skin tissue. Suitable methods are known in the art and described, for instance, in U.S. Pat. Nos. 5,489,520 and 5,550,318. See also, Potrykus (1990) Bio/Technol. 8: 535-542; and Finnegan et al. (1994) Bio/Technol. 12: 883-888.

Microinjection is a common method of nucleic acid delivery to isolated cells (Palmiter, R. D. & Brinster, R. L. Annu. Rev. Genet. 20: 465-499 (1986); Wall, R. J. et al., J. Cell Biochem. 49: 113-120 (1992); Chan, A. W. et al., Proc. Natl. Acad. Sci. USA 95: 14028-14033 (1998)). DNA is generally injected ex vivo into cells and the cells may then be re-introduced into animals. Procedures for such a technique are described in U.S. Pat. Nos. 5,175,384 and 5,434,340, and improvements to the technique are described in WO 00/69257.

Naked DNA gives virtually no transfection for cells ex vivo, but is surprisingly efficient for gene transfer in vivo following local injection. While expression of such DNA in skin only lasts for a few days, injected DNA in mouse skeletal muscle has been shown to last for up to nine months (Wolff, J. A. et al., Hum. Mol. Genet. 1: 363-369 (1992)). Naked DNA is particularly suited to gene therapy for preventive and therapeutic vaccines.

Calcium phosphate co-precipitation and electroporation are limited to ex vivo applications. Both procedures are simple, but unfortunately, while the former method is relatively inefficient, the latter results in the death of most target cells.

Cationic liposomes containing cholesterol are particularly suited for delivery of nucleic acids to humans as they are biodegradable and stable in the blood stream.

Liposomes can be injected intravenously, subcutaneously or inhaled as an aerosol (Stribling et al., 1992). Liposomes can be targeted to certain cell types by incorporating ligands, receptors or antibodies (immunolipids) into the lipid membrane (U.S. Pat. No. 4,957,773). On contacting target cells, entry of DNA from liposomes is via endocytosis and diffusion. Preparations of lipid formulations are commercially available and methods for their use are well documented (Bogdanenko, E. V. et al., Vopr. Med. Khim. 46: 226-245 (2000); Natsume, A. et al., Gene Ther. 6: 1626-1633 (1999)).

Uptake of DNA into animal cells can also be enhanced by using transfection agents. “Transfecting agent”, as utilized herein, means a composition of matter added to the genetic material for enhancing the uptake of exogenous DNA segment(s) into a eukaryotic cell, preferably a mammalian cell, and more preferably a mammalian germ cell. The enhancement is measured relative to the uptake in the absence of the transfecting agent. Examples of transfecting agents include adenovirus-transferrin-polylysine-DNA complexes. These complexes generally augment the uptake of DNA into the cell and reduce its breakdown during its passage through the cytoplasm to the nucleus of the cell. These complexes can be targeted to the male germ cells using specific ligands which are recognized by receptors on the cell surface of the germ cell, such as the c-kit ligand or modifications thereof. Other preferred transfecting agents include lipofectin, lipfectamine, DIMRIE C, Superfect, and Effectin (Qiagen), umfectin, maxifectin, DOTMA, DOGS (Transfectam; dioctadecylamidoglycylspermine), DOPE (1,2-dioleoyl-sn-glycero-3 phosphoethanolamine), DOTAP (1,2-dioleoyl-3-trimethylammonium propane), DDAB (dimethyl dioctadecylammonium bromide), DHDEAB (N,N-di-n-hexadecyl-N,N-dihydroxyethyl ammonium bromide), HDEAB (N-n-hexadecyIN, N dihydroxyethylammonium bromide), polybrene, or poly (ethylenimine) (PEI). (For example, Banerjee, R. et al., Novel series of non-glycerol-based cationic transfection lipids for use in liposomal gene delivery, J. Med. Chem. 42 (21): 4292-99 [1999]; Godbey, W. T. et al., Improved packing of poly (ethyleniminelDNA complexes increases transfection efficiency, Gene Ther. 6 (8): 1380-88 [1999]; Kichler, A et al., Influence of the DNA complexation medium on the transfection efficiency of liposperminelDNA particles, Gene Ther. 5 (6): 855-60 [1998]; Birchaa, J. C. et al., Physico-chemical characterisation and transfection efficiency of lipid-based gene delivery complexes, Int. J. Pharm. 183 (2): 195-207 [1999]). These non-viral agents have the advantage that they facilitate stable integration of xenogeneic DNA sequences into the vertebrate genome, without size restrictions commonly associated with virus-derived transfecting agents.

The most critical issues for applications such as gene therapy are the efficient delivery and appropriate expression of transgenes in host cells. For this purpose, viral systems are particularly well suited as viruses have evolved to efficiently cross the plasma membrane of eukaryotic cells and express their nucleic acids in host cells. Suitability of viral vectors is assessed primarily on their ability to carry foreign nucleic acids and deliver and express its genes with high efficiency. Current applications utilise both RNA and DNA virus based systems, and 70% of gene therapy trials use viral vectors derived from retroviruses, adenovirus, adeno-associated virus, herpesvirus and pox virus (Flotte & Carter, 1995; Glorioso et al., 1995; Smith 1995; Prince 1998; Robbins et al., 1998). Retroviruses represent the most prominent gene delivery system as they mediate high gene transfer and expression of therapeutic genes. Members of the DNA virus family such as adenovirus, adeno-associated virus or herpesvirus are popular due to their efficiency of gene delivery. Adenoviral vectors are particularly suited when transient transfection of nucleic acid is preferred. Retroviruses express particular envelope proteins that bind to specific cell surface receptors on host cells, in order for the virus to enter the cell. Hence, the type of viral vector used should be determined by the tissue type to be targeted (see e.g. Dornburg, 1995; Gunzburg, et al., 1996; Vile et al., 1996; Miller, 1997, Karavanas et al., 1998; Hu, W-S & Pathak, V. K. Pharmacol. Rev. 52: 493-511 (2000); Walther, W. & Stein, U. Drugs 60: 249-271 (2000) for reviews).

Safety is a critical issue for viral based gene delivery because most viruses are either pathogens or have pathogenic potential. Generally, when a replication-competent virus infects an animal cell it can express viral genes and release many new infectious viral particles in the host organism. Hence, it is very important that during transgene delivery the host animal does not receive a pathogenic virus with full replication potential. For this reason, viral-host cell systems have been developed for gene therapy treatments to prevent the creation of replication-competent viruses. In this method, viral components are divided between a vector and a helper construct to limit the ability of the virus to replicate (Miller 1997). The viral vector contains the gene(s) of interest and cis-acting elements that allow gene expression and replication, but contain deletions of some or all of the viral proteins. Helper cells (or occasionally, helper virus) are engineered to express the viral proteins needed to propagate the viral vectors. These new viral particles are able to infect target cells, reverse transcribe the vector RNA and integrate its DNA copy into the genome of the host, which can then be expressed. However, the vector can not express the viral proteins required to create new infectious particles. Helper cell lines are known in the art (see Hu, W-S & Pathak, V. K. Pharmacol. Rev. 52: 493-511 (2000), for a review).

In general, retroviral vectors are able to package reasonably long stretches of foreign DNA (up to 10 kb). Oncoviruses are a type of retrovirus, which only infect rapidly dividing cells. For this reason they are especially attractive for cancer therapy. Murine leukemia virus (MLV)-based vectors are the most commonly used of this class. Spleen necrosis virus (SNV), rous sarcoma virus and avian leukosis virus are other types. Lentiviral vectors are retroviral vectors that can be propagated to produce high viral titres and are able to infect non-dividing cells. They are more complex than oncoviruses and require regulation of their replication cycle. Lentiviral vectors which may be used include human immunodeficiency virus (HIV-1 and -2) and simian immunodeficiency virus (SIV) based systems. HIV infects cells of the immune system, most importantly CD4⁺ T-lymphocytes, and so may be useful for targeted gene therapy of this cell type. Another type of retrovirus is the spumavirus. Spumaviruses are attractive because of their apparent lack of toxicity (Linial 1999).

Adenoviral vectors are have high transduction efficiency and are able to transfect a number of different cell types, including non-dividing cells. They have a high capacity for foreign DNA and can carry up to 30 kb of non-viral DNA (for a review see, Kochanek, S. Hum. Gene Ther. 10: 2451-2459 (1999)). Recombinant adenoviral (rAd) vectors are becoming one of the most powerful gene delivery systems available and have been used to deliver DNA to post-mitotic neurons of the central nervous system (CNS) (Geddes, B. J. et al., Front. Neuroendocrinol. 20: 296-316 (1999), and are used to treat diseases such as colon cancer (Alvarez et al., Hum. Gene Ther. 5: 597-613 (1997). Adeno-associated virus (AAV) vectors and recombinant AAV (rAAV) vectors are proving themselves to be safe and efficacious for the long-term expression of proteins to correct genetic disease. Snyder, R. O. J. (Gene. Med. 1: 166-175 (1999)) provides a review of gene delivery approaches using such vectors. Construction of such vectors is described in, for example, Samulski et al., J. Virol. 63: 3822-3828 (1989), and U.S. Pat. No. 5,173,414.

Many gene therapy trials have been conducted and are underway (over 3,500 people have been treated with gene therapy systems), and several reviews can be studied for details of the protocols and results (Hwu & Rosenberg, 1994; Blease, 1995a,b; Breau & Clayman, 1996; Dunbar 1996; Lotze 1996). The first gene therapy trial was carried out by Blaese et al., (1995), to correct a genetic disorder known as adenosine deaminase (ADA) deficiency, which leads to severe immunodeficiency. Several cancer gene therapy strategies are being developed, which involve eliminating cancer cells by suicide therapy (Oldfield et al., 1993), modification of cancer cells to promote immune responses (Lotze et al., 1994), and reversion by delivery of a tumor suppressor gene (Roth et al., 1996). Another successful gene therapy trial has been conducted to combat graft-versus-host disease, which can result following transplant procedures such as bone marrow transplants (Bonini et al., 1997). This procedure was carried out using an HSV-based vector. Several gene therapy treatments are under investigation for the treatment of HIV-1 infection. Most treatments involve modification of lymphocytes, ex vivo, to suppress the expression of viral genes, by means of ribozymes, antisense RNA, mutant trans-dominant regulatory proteins and modification to elicit a host immune response (Nabel et al., 1994; Galpin et al., 1994; Morgan & Walker, 1996; Wong-Staal et al., 1998). Vectors currently in use for gene therapy treatments and animal tests include those derived from Moloney murine leukemia virus, such as MFG and derivative thereof, and the MSCV retroviral expression system (Clontech, Palo Alto, Calif.). Many other vectors are also commercially available.

Viral vectors are especially important in applications when a specific tissue type is to be targeted, such as for gene therapy applications. There are two available methods for targeting genes to specific cell or tissue types. One strategy is designed to control expression of the required gene using a tissue specific promoter (discussed above), and another strategy is to control viral entry into cells. Viruses tend to enter specific cell types according to the envelope proteins that they express. However, by engineering the envelope proteins to express specific proteins as fusions, such as erythropoietin, insulin-like growth factor I and single chain variable fragment antibodies, viral vectors can be targeted to specific cell-types (Kasahara et al., 1994; Somia et al., 1995; Jiang et al., 1998; Chadwick et al., 1999).

In one example of tissue specific targeting in transgenic mice, a novel transgene delivery system has been developed in which the target tissue type expresses an avian viral receptor (TVA), under the control of a tissue specific promoter. Transgenic mice expressing the TVA receptor are then infected with avian leukosis virus, carrying the transgene(s) of interest (Fisher, G. H. et al., Oncogene 18: 5253-5260 (1999).

### EXAMPLES

The present invention will now be described by way of the following examples, which are illustrative only and non-limiting. In the Examples below, we describe several specific embodiments of the invention. In one embodiment, we present a zinc finger polypeptide containing a structured linker, TFIIIAZif fused to the VP64 activation domain, which activates the expression of a reporter construct integrated into the genome of a transgenic animal. In another embodiment, we present a zinc finger polypeptide comprising two 3-finger domains joined by a flexible linker which is able to up-regulate the expression of an endogenous gene in an animal. In yet another embodiment, we present a zinc finger polypeptide comprising two 3-finger domains joined by a flexible linker which is able to down-regulate the expression of an endogenous gene in an animal.

The Examples show that a zinc finger polypeptide can be expressed in animals and recognises a target DNA sequence in an animal genome. Secondly, the Examples show that zinc finger polypeptides containing a transactivating domain can activate the expression of a target gene in animals in a manner analogous to that of endogenous zinc finger proteins in animal cells. Using this principle and the consensus methods described herein, zinc finger polypeptides can be designed to interact with specific target nucleotide sequences to either activate or repress the expression of target genes.

It will be appreciated that the zinc finger polypeptides shown here may further comprise one or more effector domains. Furthermore, it will be clear that other embodiments are possible, and that the Examples should not be taken as limiting.

### Example 1

### Zinc Finger Gene Construction and Cloning

In general, procedures and materials are in accordance with guidance given in Sambrook et al. Molecular Cloning. A Laboratory Manual, Cold Spring Harbor, 1989.

a. Construction of Zinc Finger Polypeptide

The gene encoding the Zif268 zinc finger polypeptides (residues 333-420) is assembled from 8 overlapping synthetic oligonucleotides, giving SfiI and NotI overhangs (Choo and Klug (1994)). The genes encoding zinc finger polypeptides of the phage library are synthesized from 4 oligonucleotides by directional end-to-end ligation using 3 short complementary linkers, and amplified by PCR from the single strand using forward and backward primers which contain sites for NotI and SfiI respectively. Backward PCR primers in addition introduce Met-Ala-Glu as the first three amino acid residues of the zinc finger polypeptides, and these are followed by the residues of the wild type or library zinc finger polypeptides as required. Cloning overhangs were produced by digestion with SfiI and NotI where necessary. Nucleic acid encoding zinc finger polypeptide fragments were ligated into similarly prepared Fd-Tet-SN vector. This is a derivative of fd-tet-DOG1 (Hoogenboom et al. (1991) Nucl. Acids Res. 19:4133-4137), in which a section of the pelB Leader and a restriction site for the enzyme SfiI (underlined) have been added by site-directed mutagenesis using the oligonucleotide:

5′ CTCCTGCAGTTGGACCTGTGCCATGGCCGGCTGGG
CCGCATAGAATGGAACAACTAAAGC 3′

that anneals in the region of the polylinker. Electrocompetent DH5α cells were transformed with recombinant vector in 200 ng aliquots, grown for 1 hour in 2×TY medium with 1% glucose, and plated on TYE containing 15 μg/ml tetracycline and 1% glucose.

Construction of Zinc Finger Polypeptide for Reporter Assays

The zinc finger polypeptide used for this first set of experiments is a fusion protein that comprises 4 domains. First, the first 4 fingers of TFIIIA are fused N-terminally to the 3 fingers of Zif268, using standard PCR procedures, and the construct is denoted TFIIIAZif. These peptides are fused from the last amino acid of the linker separating fingers 4 and 5 of TFIIIA, to the first residue of the N-terminal finger of Zif268 (Choo & Klug (1997) Curr. Opin. Str. Biol. 7:117-125; Pavletich & Pabo (1991) Science 252:809-817; Elrod-Erickson et al. (1996) Structure 4:1171-1180; and Elrod-Erickson et al (1998) Structure 6:451-464).

TFIIIAZif

MGEKALPVVYKRYICSFADCGAAYNKNWKLQAHLCKHTGEKPFPCKEEGC
EKGFTSLHHLTRHSLTHTGEKNFTCDSGCDLRFTTKANMKKHFNRFHNIK
ICVYVCHFENCGKAFKKHNQLKVHQFSHTQQLPYACPVESCDRRFSRSDE
LTRHIRIHTGQKPFQRCICMRNFSRSDHLTTHIRTHTGEKPFACDICGRK
FARSDERKRHTKIHLRQKD

This designed zinc finger polypeptide is able to recognize specifically a DNA sequence of 27 base pairs (bp), which comprises the 11 bp binding site of TFIIIA fingers 1-3, and the 9 bp target site of Zif268, separated by a 7 bp spacer (binding sites are shown in bold).

5′ GCGTGGGCG TGTACCT GGATGGGAGAC 3

The second domain is the 7 amino acid nuclear localisation sequence (NLS) of the wild-type Simian Virus 40 large-T antigen (Kalderon et al., Cell 39:499-509 (1984), which was fused to the C-terminus of the zinc finger polypeptide, to direct the chimeric polypeptide to the nucleus. Third, a tetramer of the transactivation domain from the Herpes Simplex Virus (HSV), VP64 (or VP16, which is the minimal transactivation domain) is fused to the construct. The fourth domain is the 9E10 region that corresponds to a myc epitope tag, and allows the specific antibody recognition of the expressed zinc finger polypeptide in animals, if required. This region is fused to the extreme C-terminus of the chimeric polypeptide.

The sequence of the SV40-NLS-VP64-c-myc repressor domain (NLS-VP64-c-myc domain sequence) is as follows (N- to C-terminal):

AARNSGPKKKRKVELQLTSDALDDFDLDMLGSDALDDFDLDMLGSDALDD
FDLDMLGSDALDDFDLDMLSSQLSQEQKLISEEDL

Construction of Zinc Finger Polyeptides for Endogenous Gene Regulation

To target any nucleotide sequence in a transgenic animal, zinc finger polypeptide phage display libraries are made and used for selections against the desired nucleotide sequence, as described in our patent publication WO 98/53057. The phage display library contains amino acid randomisations of the putative base-contacting positions in the first and second, or second and third fingers of the three-finger DNA binding domain of Zif268, and hence, contains members that bind DNA of the sequence GCGGXXX, or XXXXGGCG, respectively, where X is any base. After this initial selection protocol selected finger domains are be recombined to generate three-finger peptides which recognise the desired 9 or 10 base nucleotide region (for further details refer to WO 98/53057).

Zinc finger engineering using this system can be completed in less than two weeks and yields three-zinc finger polypeptide molecules that bind sequence-specifically to DNA with affinities in the nanomolar range. Three-finger zinc finger polypeptides selected (according to WO 98/53057) to bind specific 9 (or 10) base nucleotide sequences within the same target sequence are fused together to create high-affinity six-finger peptides. The resulting six-finger peptides are able to target virtually unique 18 bp nucleotide stretches within any animal cell, giving the potential for specific regulation of any target gene, as described above.

Zinc Finger Polypeptide for Repression of Mouse TNFR1 Gene

Using the procedures described above and detailed in our patent publication (WO98/53057), two 3-finger domains are selected to bind the promoter of the mouse TNFR1 gene (see Kemper, O. & Wallach, D. Gene 134: 209-216 (1993)). The region of the mouse TNFR1 promoter sequence targeted is about 250 bp upstream of the putative transcriptional start site. The sequence of this region is shown below, with the exact bases targeted indicated in bold.

5′ AGTGGTGTTAAGTGGGTTTGGGGCGCCAAGCT 3′

Having thus generated 3-finger peptides to bind the continuous 9 bp sequences TTAAGTGGG and TTTGGGGCG, the 3-finger units are then fused together with a flexible linker of the sequence (N- to C-terminus): TGSERP, to create a 6-finger polypeptide with the 18 bp DNA recognition sequence shown above, termed TNFR1-M4-2.

The amino acid sequences of the helical regions from the TNFR1-M4-2 polypeptide are shown in Table 1 below. Residues are numbered relative to the first position in the α-helix (position 1) in each finger (F1-6).

TNFR1-M4-2 (Linker TGSERP Between F3 and F4)

TABLE 1
TNFR1-M4-2 Binding Sequences

| F1 | F2 | F3 | F4 | F5 | F6 |
|---|---|---|---|---|---|
| −1123456 | −1123456 | −1123456 | −1123456 | −1123456 | −1123456 |
| RSADLTR | RRDHLSE | TNDSRTN | RSQHLTE | TSSHLSK | QSNARKT |

The TNFR1-M4-2 polypeptide is then engineered into a transcriptional repression polypeptide to down-regulate the expression of the mouse TNFR1 gene. The repressor construct contains the zinc finger DNA binding domain TNFR1-M4-2 at the N-terminus, fused in frame to the translation initiation sequence ATG. The 7 amino acid nuclear localisation sequence (NLS) of the wild-type Simian Virus 40 large-T antigen (Kalderon et al., Cell 39:499-509 (1984)) is fused to the C-terminus of the zinc finger sequence and a repressor domain, such as the Kruppel-associated box (KRAB) repressor domain from human KOX1 protein (Margolin et al., Proc. Natl. Acad. Sci. USA 91:4509-4513 (1994)), the engrailed domain (Han et al., EMBO J. 12: 2723-2733 (1993)) or the snag domain (Grimes et al., Mol Cell. Biol. 16: 6263-6272 (1996)), is fused downstream of the NLS.

The KOX1 domain contains amino acids 1-97 from the human KOX1 protein (database accession code P21506) in addition to 23 amino acids which act as a linker. In addition, a 10 amino acid sequence from the c-myc protein (Evan et al., Mol. Cell. Biol. 5: 3610 (1985)) is introduced downstream of the KOX1 domain as a tag to facilitate expression studies of the fusion protein.

The complete amino acid sequence of the zinc finger chimeric repressor polypeptide, TNFR1-M4-2-Kox1, is shown below:

MAERPYACPVESCDRRFSRSADLTRHIRIHTGQKPFQCRICMRNFSRRDH
LSEHIRTHTGEKPFACDICGRKFATNDSRTNHTKIHTGSERPYACPVESC
DRRFSRSQHLTEHIRIHTGQKPFQCRICMRNFSTSSHLSKHIRTHTGEKP
FACDICGRKFAQSNARKTHTKIHLRQKDAARNSGPKKKRKVDGGGALSPQ
HSAVTQGSIIKNKEGMDAKSLTAWSRTLVTFKDVFVDFTREEWKLLDTAQ
QIVYRNVMLENYKNLVSLGYQLTKPDVILRLEKGEEPWLVEREIHQETHP
DSETAFEIKSSVEQKLISEEDL

The amino acid sequence of the zinc finger domain is displayed in bold, and that of the SV40-NLS-KOX1-c-myc repressor domain is in normal type (N- to C-terminal).

B. Zinc Finger Polypeptide for Activation of Mouse Erythropoietin Gene

Using the procedure described above and detailed in our patent publication (WO98/53057), two 3-finger domains are selected to bind the promoter of the mouse erythropoietin gene (see Shoemaker, C. B. & Mitsock, L. D. Mol. Cell. Biol. 6: 849-858 (1986), and Beru, N. et al., DNA 8: 253-259 (1989)). The region selected is approximately 950 bp upstream of the transcriptional start point, and the sequence of that region is shown below, with the 9 bp target sites indicated in bold:
5′ CCCCCAGTGAGGGGCTGGGGGTGTGGCTCAG 3′

Using standard PCR techniques, the 3-finger domains selected to bind the 9 bp sites: GGTGTGGGG and GTCGGGGAG are joined to create a 6-finger polypeptide, using the linker sequence (N- to C-terminus): TGSERP, between the third and fourth fingers. The resulting 6-finger polypeptide, called EPO-M10-9 binds specifically to the 18 bp target sequence shown above. The amino acid sequences of the helical regions from the EPO-M10-9 polypeptide are displayed in Table 2 below. Residues are numbered relative to the first position in the -helix (position 1) in each finger (F1-6).

TNFR1-M4-2 (Linker TGSERP Between F3 and F4)

TABLE 2
EPO-M10-9 Binding Sequences

| F1 | F2 | F3 | F4 | F5 | F6 |
|---|---|---|---|---|---|
| −1123456 | −1123456 | −1123456 | −1123456 | −1123456 | −1123456 |
| RSSHLST | RSDTLTR | RNDHRTK | RSDALSE | RNSHRTK | RSDNLTR |

The EPO-M10-9 polypeptide is then engineered into a transcriptional activator protein, in a similar manner as described for the TNFR1-M4-2 construct above, except that the KOX1 domain is substituted for the VP64 (or VP16) activation domain of HSV, or another suitable activation domain. The resulting transcriptional activation peptide is called EPO-M10-9-VP64, and has the sequence shown below.

MAERPYACPVESCDRRFSRSADLTRHIRIHTGQKPFQCRICMRNFSRRDH
LSEHIRTHTGEKPFACDICGRKFATNDSRTNHTKIHTGSERPYACPVESC
DRRFSRSQHLTEHIRIHTGQKPFQCRICMRNFSTSSHLSKHIRTHTGEKP
FACDICGRKFAQSNARKTHTKIHLRQKDAARNSGPKKKRKVELQLTSDAL
DDFDLDMLGSDALDDFDLDMLGSDALDDFDLDMLGSDALDDFDLDMLSSQ
LSQEQKLISEEDL

The amino acid sequence of the zinc finger domain is displayed in bold, and that of the SV40-NLS-VP64-c-myc repressor domain is in normal type (N- to C-terminal).

b. Cloning of Zinc Finger Polypeptides for Expression in T-Cells

Expression cassettes for TFIIIAZif-NLS-VP64-c-myc, TNFR1-M4-2-Kox1, and EPO-M10-9-VP64 constructs are created in a similar fashion.

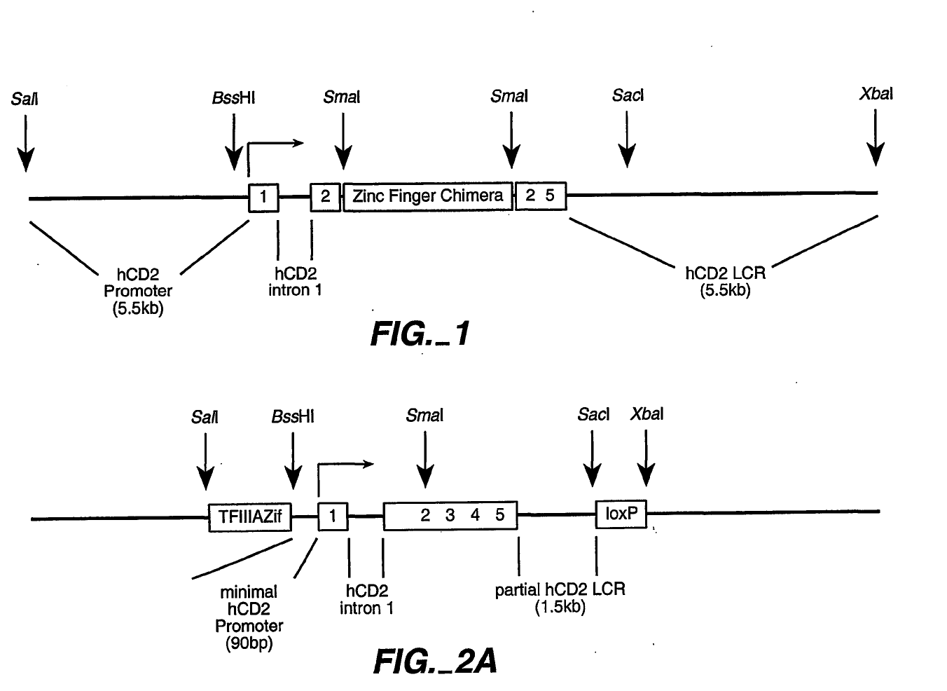

First all zinc finger chimeric polypeptide genes, (immediately followed by a stop codon) are inserted into the multiple cloning site of the pcDNA3.1(−) vector (Invitrogen) between the XbaI and BamHI sites. The expression cassettes are derived from the expression vector VA (MI51), which is a customised version of pbluescript SK(−) from Stratagene (Zhumabekov, T. et al., J. Immun. Methods 185: 133-140 (1995)). This vector contains the human CD2 (hCD2) gene promoter, which gives activity only in the T-lymphocyte lineage and the hCD2 locus control region (LCR), which ensures copy number-dependent, position independent expression in this cell type. Lying between the promoter and LCR sequences, the vector contains exons 1, 2 and 5 of hCD2, with intron 1 of the gene between exons 1 and 2. The presence of the intron is thought to give better expression of associated transcripts in vivo (Festenstein, R. et al. 1996 Science 271: 1123). The zinc finger genes are excised from pcDNA3.1(−) using the PmeI site at each end of the multiple cloning site, and this PmeI fragment is then blunt-ended by treatment with the Klenow fragment. The VA vector construct is digested with SmaI, which cuts within the second exon of the hCD2 gene, giving blunt ends. Finally, the blunt ended fragments containing the zinc finger chimeric polypeptide genes for TFIIIAZif-NLS-VP64-c-myc, TNFR1-M4-2-Kox1, and EPO-M10-9-VP64 are ligated into the VA vector and sequenced to select plasmids containing the zinc finger genes in the correct orientation. These constructs are called MITFIIIAZif, MITNFR1 and MIEPO (see FIG. 1).

### Example 2

### Reporter Gene Construction and Cloning

The reporter constructs described are based on the human CD2 gene and the destabilised enhanced green fluorescent protein (EGFP). However, any other suitable reporter gene such as β-galactosidase and β-lactamase may be used instead.

Reporter Construct for Expression in T-Cells

Two reporter constructs are created for expression studies in T-cells, which are based on the vectors reported by Festenstein, R. et al. (Science 271: 1123 (1996)). The first is a mini-gene construct consisting of the 5 exons of hCD2, with intron 1 of hCD2 between exons 1 and 2. The second reporter construct is the same as the first, except it also contains intron 4 of hCD2 between exons 4 and 5. Both gene constructs are positioned between the promoter and LCR of hCD2. These reporter constructs are known as hCD2 “minigene” constructs (see Festenstein, R. et al. 1996 Science 271: 1123). To make the expression of hCD2 from these vectors dependent on activation by TFIIIAZif, the hCD2 promoter is modified to create a minimal promoter with binding sites for the TFIIIAZif polypeptide. First, the construct is digested from pBluescript SK(−) with the restriction endonucleases XbaI and BssHII. BssHII cuts 90 bp upstream of the transcriptional start site of hCD2 and so the restriction fragment lacks the first 5.4 kilobase pairs (kb) of the hCD2 promoter. The resultant 90 bp of the hCD2 promoter is a minimal promoter, which gives low but detectable activity of the hCD2 gene in vivo. TFIIIAZif binding sites are constructed by annealing complimentary oligonucleotides (A with D, B with E, C with F) which create 1, 2, or 3 copies of the TFIIIAZif binding site, respectively, each separated by 6 bp (as shown below; binding sites are shown in bold):

TCGAC (TATGCGTGGGCGTGTACCTGGATGGGAGACCG)_{N}G
(N = 1, Primer A; N = 2, Primer B; N = 3,
Primer C)
CGCGC (CGGTCTCCCATCCAGGTACACGCACCCGCATA)_{X}G
(N = 1, Primer D; N = 2, Primer E; N = 3,
Primer F)

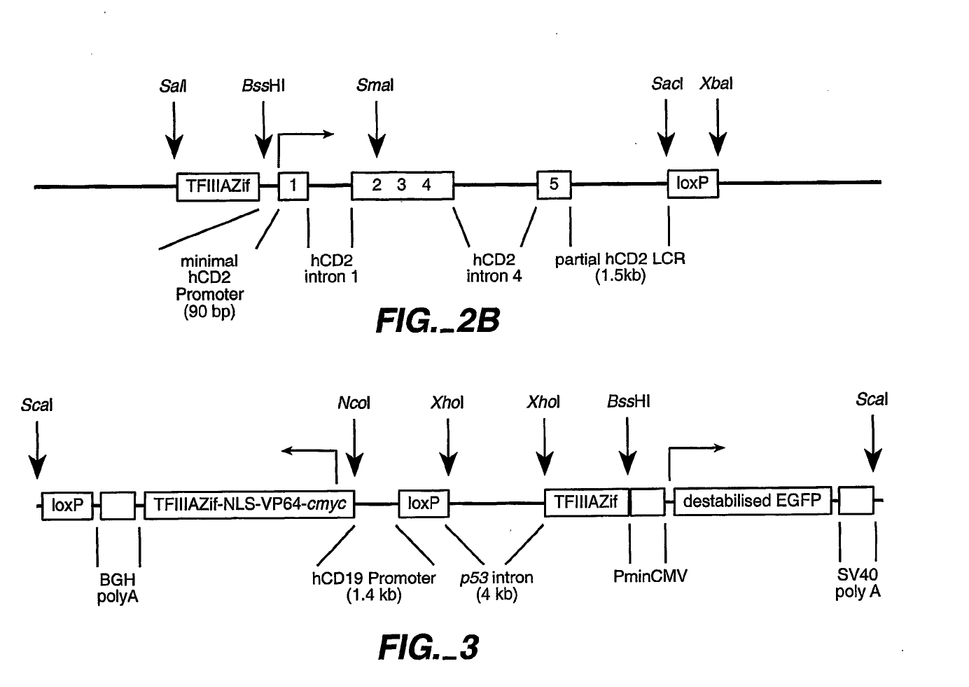

The annealed oligonucleotides also generate BssHII and SalI restriction ends. These TFIIIAZif binding site-containing DNA fragments can be ligated to the 5′ end of the reporter construct such that they are positioned immediately upstream of the minimal promoter. Next, a partial LCR of the hCD2 gene is created, by digesting the minigene construct with the restriction endonuclease SacI. SacI cleaves 1.5 kb into the hCD2 LCR, thereby removing 4 kb of the LCR from the 3′ end of the XbaI, BssHII fragment. The partial LCR does not retain full activity and therefore the reporter transgene is subject to increased position effect variegation (Zhumabekov, T. et al. 1999, EMBO J. 18: 6396-6406). Finally, a single loxP recombination signal sequence (for Cre recombinase, see above) is inserted at the 3′ end of the partial LCR. The loxP site is produced by annealing the complimentary oligonucleotides G and H, which also generate SacI and XbaI ends. The double stranded loxP site is ligated to the 3′ end of the new minigene construct using the SacI restriction ends, and the complete SalI, XbaI fragments are inserted into SalI, XbaI cut pBluescript SK(−). The constructs with only intron 1 are known as MICD2-1, -2, or -3 and those also containing intron 4 are known as MI4CD2-1, -2, -3 according to the number of TFIIIAZif binding sites preceding the reporter gene (see FIG. 2). By mating mice containing tandem repeats of the reporter with a transgenic mouse containing a suitably expressed Cre recombinase, reduction down to a single copy of the reporter gene is possible through the use of the single loxP site. This facilitates the production of mouse strains with single copy transgenes.

(Primer G)
C [symbol] ATGTATGC [symbol] T
(Primer H)
CTAGA [symbol] GCATACAT [symbol] GAGCT

b. Reporter Construct for Expression in B-Cells

A second transgene construct is created in which the TFIIIAZif-NLS-VP64-c-myc and reporter genes are contained on the same DNA molecule. This eliminates the additional step of having to crossbreed transgenic mouse lines. The zinc finger effector gene is under the control of a B-cell specific promoter, the human CD19 promoter (sequence can be found in GenBank, accession no. M84371). The reporter gene is a destabilised version of EGFP and is cloned in an anti-sense orientation with respect to the TFIIIAZif-NLS-VP64-c-myc expression cassette. The EGFP gene is under the sole control of a TFIIIAZif-dependent promoter, placed immediately upstream of the EGFP gene. An intron derived from the human p53 gene is inserted between the zinc finger and EGFP genes. This intron acts as a transcriptional insulator, to further prevent ‘leakage’ between the effector and reporter genes (Utomo et al., Nat. Biotech. 17: 1091-1096 (1999)). The CD19 promoter and the gene for TFIIIAZif-NLS-VP64-c-myc are flanked by loxP sites to give the option of removing the zinc finger polypeptide, by crossing with an appropriate mouse strain expressing Cre recombinase, to provide a negative control for EGFP expression.

TFIIIAZif-NLS-VP64-c-myc is cloned into pcDNA3.1(−) as above (Example 1b), and extracted by PCR using primers I and J. The PCR fragment contains the TFIIIAZif-NLS-VP64-c-myc gene, operably linked to the bovine growth hormone (BGH) poly-adenylation sequence from the pcDNA3.1(−) vector, at the 3′ end. The primers also add a NcoI restriction site at the position of the first methionine residue of TFIIIAZif at the 5′ end, and a loxP site and ScaI site at the 3′ end of the zinc finger gene (Primers I and J, respectively; restriction sites are underlined, loxP sites are shown in bold italics, PCR annealing sequences are shown in bold).

(Primer I)
CTACGCCCATGGGAGAGAAGGCGCTGCCGG
(Primer J)
CTAGCAGTACTC [symbol] GCATACAT [symbol]
CCAGAATAGAATGACACCTACTCAGAC

A 1.4 kb fragment of the human CD19 promoter sequence, immediately upstream of the CD19 gene, was amplified by PCR from purified genomic DNA using primers K and L, which create a loxP site and XhoI restriction site at the 5′ end and a NcoI restriction site at the 3′ end (Primers. K, L respectively; restriction sites are underlined, loxP sites are shown in bold italics, PCR annealing sequences are shown in bold)

(Primer K)
CTACGCCTCGAG [symbol] ATGTATGC [symbol] GGAT
CCTCTCGCCTCGGCCTCC
(Primer L)
TACCTACCATGGTGGTCAGACTCTCCGGGG

The PCR primers generate NcoI sites at the position of the first methionine residue of TFIIIAZif, and at the equivalent point in the CD19 promoter/gene sequence. Hence, by joining the zinc finger gene to the CD19 promoter PCR fragment, the TFIIIAZif construct is operably linked to the human CD19 promoter. The destabilised EGFP gene, along with an operably linked minimal promoter from the human cytomegalovirus (P_{minCMV}), and an SV40 polyadenylation signal is extracted from the vector pTRE-dEGFP (Contech), by PCR using the primers M and N. These primers add a BssHII site at the 5′ end of the minimal CMV promoter and a ScaI restriction site at the 3′ end of the construct (Primers M and N, respectively; restriction sites are underlined, PCR annealing sequences are shown in bold).

(Primer M)
GACTATGCGCGCGTACCCGGGTCGAGTAGGCGTG
(Primer N)
TAGGCTAGTACTCACACCTCCCCCTGAACCTGAAAC
TCGAG (TATGCGTGGGCGTGTACCTGGATGGGAGACCG)_{N}G
(N = 1, Primer O; N = 2, Primer P; N = 3,
Primer Q)
CGCGC (CGGTCTCCCATCCAGGTACACGCACCCGCATA)_{X}C
(X = 1, Primer R; X = 2, Primer S; X = 3,
Primer T)

One to three binding sites for the TFIIIAZif polypeptide are created by annealing complimentary oligonucleotides: Primer O with Primer R; Primer P with Primer S; and Primer Q with Primer T, which also create XhoI and BssHII restriction ends, and these are fused to the minimal CMV promoter at the BssHII site. The reporter construct (fused to the TFIIIAZif binding sites), and the effector gene (under the control of the human CD19 promoter), are digested with XhoI and ligated together. This DNA fragment is then cut with ScaI and ligated into similarly cut pAU7-28 (Utomo, A. R. H. et al., Nat. Biotech. 17: 1091-1096 (1999)), to generate pAU7-p53. Finally, the 4 kb XhoI fragment of the human p53 intron (provided by E. Bockamp, Mainz, Germany) is ligated into the XhoI site of this vector, to generate, pATFIIIAZif-1, -2 or -3, depending on the number of TFIIIAZif binding sites preceding the reporter (see FIG. 3). Correct constructs are confined by standard sequencing and restriction digestion.

### Example 3

### Creation and Screening of Transgenic Mice

Expression Constructs in T-Lymphocytes

The reporter and zinc finger chimeric polypeptide expression vectors, MITFIIIAZif, MITNFR1, MIEPO, MICD2-1, -2 and -3 and MI4CD2-1, -2, and -3 are linearised by digestion with SalI and NotI and the inserts containing reporter or effector genes are purified. These linear DNA fragments are microinjected into the pronuclei of fertilised mouse cells, and re-implanted into the oviduct of a recipient female, using standard procedures known to those with skill in the art (see above, and Gordon, J. & Ruddle, F. H. Science 214: 1244-1246 (1981); Gordon; J & Ruddle, F., Methods in Enzymology 101: 411-433 (1983); Hogan et al., Manipulating the Mouse Embryo: A Laboratory Manual (1988)). This creates transgenic mice containing either a zinc finger polypeptide expression cassette, or a reporter construct. To create transgenic mice expressing hCD2 in T-lymphocytes, transgenic mice containing the gene for TFIIIAZif-NLS-VP64-c-myc, under the control of the hCD2 promoter and LCR, are crossed with transgenic mice containing the hCD2 reporter construct. The F1 progeny of the above mating now carry both the effector and reporter constructs, and express TFIIIAZif-NLS-VP64-c-myc specifically in T-lymphocytes.

Southern blotting and PCR analysis using TFIIIAZif or hCD2 specific primers and probes are used to identify transgenic progeny and to estimate the copy number of incorporated transgenes. The procedures used are standard and known to those in the art, see U.S. Pat. No. 4,683,202, and Erlich et al., Science 252: 1643 (1991)).

Expression Constructs in B-Cells

As in Example 3a, above, the vector containing the TFIIIAZif activator polypeptide, and the destabilised EGFP reporter, pATFIIIAZif, must be linearised before microinjection. Therefore, pATFIIIAZif is digested with ScaI to linearise it, and the DNA containing the zinc finger and reporter genes is microinjected into the pronuclei of fertilised mouse cells and treated as described above.

### Example 4

### Expression of hCD2 in an Animal

T-cells are isolated from the thymus or lymph nodes of F1 mice containing both TFIIIAZif-NLS-VP64-c-myc transactivator and hCD2 reporter genes, according to standard surgical techniques. The TFIIIAZif-NLS-VP64-c-myc polypeptide is detected by standard Western blotting and immuaohistochemical procedures, using an anti-c-myc antibody. The DNA-binding activity of the TFIIIAZif-NLS-VP64-c-myc polypeptide can also be measured by EMSA with nuclear extracts from T-lymphocytes (see Moore, N. C., Girdlestone, J., Anderson, G., Owen, J. J. T., Jenkinson, E. (1995) J. of Immunology 155: 4653-4660).

Standard RT-PCR and Northern blotting procedures are used to demonstrate up-regulation of the hCD2 transgene in response to TFIIIAZif-NLS-VP64-c-myc, using hCD2 gene specific primers and probes, as shown (Primers. U, V, W):

| | | |
|---|---|---|
| Forward: | 5′ CCAGCCTGAGTGCAAAATTCA 3′ | (Primer U) |
| Reverse: | 5′ CAGGCTCGACACTGGATTCC 3′ | (Primer V) |
| Probe: | 5′ TGCTGACTTTGTTCCCTGCTGTGCA 3′ | (Primer W) |

RNA is isolated from approximately 1×10⁶ cells using the RNeasy RNA Isolation Kit (Qiagen) according to the manufacturer's instructions. The amount of total RNA is determined by absorbance at 260 nm. cDNA is transcribed using Superscript™ First-Strand Synthesis System for RT-PCR (GibcoBRL Life-Tech) using random hexamers as primers, according to the manufacturers instructions. Primers and probe specific for hCD2 mRNA were created using Primer Express Software (PE Applied Biosystems, UK). The probe is labelled 5′ with FAM (6-carboxyfluorescein) and 3′ with TAMRA (6-carboxytetramethylrhodamine). Quantification of mRNA was carried out on an ABI Prism 7700 Sequence Detection System (PE Applied Biosystems, UK) as instructed by the manufacturer.

Additionally, the presence of hCD2 on the surface of T-lymphocytes isolated from negative control and TFIIIAZif-NLS-VP64-c-myc containing transgenic mice is detected using a monoclonal anti-hCD2 antibody, using standard cytofluorimetric procedures.

The above analyses are also carried out on transgenic mice that contain the hCD2 transgene, but not the TFIIIAZif-NLS-VP64-c-myc effector polypeptide. These mice act as negative controls for the transactivation of the reporter construct. The results demonstrate the transactivation of the hCD2 reporter gene by the heterologous zinc finger polypeptide in an animal.

### Example 5

### Expression of EGFP in an Animal

The CD19 B cell-specific promoter is used to drive expression of a cDNA encoding the TFIIIAZif-NLS-VP64-c-myc polypeptide in mouse B-cells. In negative control mice, the cDNA encoding the 1.4 kb CD19 promoter and the TFIIIAZif-NLS-VP64-c-myc gene are excised by crossing the transgenic mice with a strain expressing Cre recombinase, as detailed above.

Lymph-node derived B-cells were isolated using standard surgical procedures. Western blotting, immunohistochemical assays, and EMSA are carried out (as above), to analyse the expression and binding activity of the effector polypeptide.

Standard RT-PCR and Northern blotting procedures are used to demonstrate up-regulation of the EGFP transgene in cells also expressing the TFIIIAZif-NLS-VP64-c-myc polypeptide, using EGFP gene specific primers and probes, shown below (Primers X, Y, Z). Again, the probe is labelled at its 5′ with FAM (6-carboxyfluorescein) and at its 3′ with TAMRA (6-carboxytetramethylrhodamine).

| | | |
|---|---|---|
| Forward: | 5′ AGCAAAGACCCCAACGAGAA 3′ | (Primer X) |
| Reverse: | 5′ GGCGGCGGTCACGAA 3′ | (Primer Y) |
| Probe: | 5′ CGCGATCACATGGTCCTGCTGG 3′ | (Primer Z) |

Further, EGFP expression is assayed by cytofluorimetry on B-cells from test and negative control mice, to demonstrate the TFIILIAZif-NLS-VP64-c-myc polypeptide specific activation of the EGFP reporter gene in a transgenic mouse.

### Example 6

### Down-Regulation of an Endogenous Mouse Gene

To determine whether a suitably configured zinc finger polypeptide could be used to repress gene transcription from an endogenous gene in an animal, the mouse TNFR1 gene was selected as a target. TNFR1 (CD120a) and TNFRII (CD120b) both act as cell-surface receptors for the signalling molecule, TNFα (Chan, F. K., Siegel, R. M., Lenardo, M. J., Signaling by the TNF Receptor Superfamily and T Cell Homeostasis. Immunity 13: 419-422 (2000)). TNFα serves an important function in promoting inflammation in order to neutralise pathogens, but it is often associated with a range of clinical problems (Immunology. Eds Roitt, I., Brostoff, J., Male, D. Mosby, London, 4^{th} edition (1996); Kollias, G., Douni, E., Kassiotis; G., Kontoyiannis, D. Immunol Rev. 169: 175-194 (1999)). For example, acute over-production of TNFα in response to bacterial toxins can cause septicaemia, toxic shock syndrome, and other forms of immune damage. Chronic autoimmune diseases and other syndromes including inflammatory bowel disease, rheumatoid arthritis, psoriasis, myocarditis, myelodysplasia, multiple sclerosis, and type II diabetes are also linked to TNFα. Murine models have shown that over-expression of TNFα can lead to myocardial fibrosis, and this could be ameliorated with adenoviral gene therapy with a decoy TNF receptor (Li, Y. Y., Feng, Y. Q., Kadokami, T., Mctiernan, C. F., Draviam, R., Watkins, S. C., Feldman, A. M. Proc. Natl. Acad. Sci. USA 97: 12746-12751 (2000)). The pivotal role of TNFα in rheumatoid arthritis is illustrated by the favourable clinical responses of patients to treatment with an antibody to TNFα, Infliximab (Maini, R. N., Taylor, P. C., Paleolog, E., Charles, P., Ballara, S., Brennan, F. M., Feldmann, M. Ann. Rheum. Disease 58: 156-160 (1999)), or a recombinant decoy receptor, Etanercept (Garrison, L., McDonnell, N. D. Ann. Rheum. Disease 58:165-169 (1999)).

TNFR1 and TNFRII have distinct immunological functions, as found in studies of mouse strains where genes for one or both have been knocked-out. Mouse strains susceptible to myocarditis do not develop inflammatory heart disease when TNFR1 is not expressed but TNFRII is still present (Bachmaier, K., Pummerer, C., Kozieradzki, I., Pfeffer, K., Mak, T. W., Neu., N., Penninger, J. M. Circulation 95: 655-661 (1997)). Similarly, in a murine model of experimental autoimmune encephalomyelitis (EAE), knock-out of TNFRI prevented EAE, while knockout of TNFRII exacerbated the disease (Suvannavejh, G. C., Lee, H. O., Padilla, J., Dal Canto, M. C., Barret, T. A., Miller, S. D. Cell. Immunology 205: 24-33 (2000)). Thus, repression of the TNFR1 gene may give important therapeutic benefits to many human conditions.

The DNA sequence of the regulatory region, immediately 5′ to the mouse TNFR1 gene was used to select potential binding sites for engineered zinc finger polypeptides. Zinc finger polypeptides to specifically bind to this promoter region are engineered according to the method of WO 98/53057, and chimeric repressors are made as described above.

The expression cassette for the TNFR1-M4-2-Kox1 polypeptide is created by operably linking the nucleic acid encoding the zinc finger effector protein between the hCD2 promoter and LCR, as described in Example 1b, above.

Transgenic mice expressing the TNFR1-binding zinc finger repressor polypeptide are created by microinjecting the SalI, XbaI linearised plasmid (MITNFR1) containing the effector gene, into the pronuclei of fertilised eggs and re-implanting the eggs into a female mouse, as described in example 2. Progeny are screened by Southern analysis and standard PCR techniques to determine which are transgenic mice. Thymocytes and T-cells are then isolated from mice containing the zinc finger repressor polypeptide, and negative control mice, according to standard surgical techniques.

The expression of the zinc finger polypeptide can be analysed, as before, using standard Western blotting and immunohistochemical procedures, using an anti-c-myc antibody.

The level of mouse TNFR1 mRNA is assayed by standard procedures of RT-PCR and Northern blotting, using mouse TNFR1 sequence specific primers and probes, created as explained in Example 4, to determine the amount of transcriptional activity from the endogenous TNFR1 gene.

Additionally, the levels of mouse TNFR1 protein expressed in the T-cells can be determined using immunohistochemical staining with an anti-mouse TNFR1 antibody.

### Example 7

### Up-Regulation of an Endogenous Mouse Gene

Many symptoms associated with kidney failure are frequently due to anaemia and are refractory to kidney dialysis. Anaemia leaves dialysis patients fatigued and exhausted, impairing their ability to work or perform even routine tasks. This is caused by insufficient production of erythropoietin (EPO), a protein naturally produced by functioning kidneys, which circulates through the bloodstream to the bonemarrow, stimulating the production of red blood cells. Administration of recombinant EPO increases the haematocrit of sufferers and restores their ability to lead a normal life. EPO is naturally secreted from the cells in which it is produced, therefore, by expressing EPO in cells which do not normally produce this protein, such as T-lymphocytes, the normal balance of EPO in the blood stream could be recovered in anaemic patients. Hence, the mouse EPO gene was selected as a target to determine whether a suitably configured zinc finger polypeptide can be used to activate gene expression from an otherwise silent endogenous gene in an animal.

The DNA sequence of the regulatory region, 5′ to the mouse EPO gene was used to select potential binding sites for engineered zinc finger polypeptides. Zinc finger polypeptides to specifically bind to this promoter region are engineered according to the method of WO 98/53057, and chimeric activator proteins are made as described above.

The expression cassette for the EPO-M10-9-VP64 polypeptide is created by operably linking the nucleic acid encoding the zinc finger effector protein between the hCD2 promoter and LCR, as described in Example 1b, above.

Transgenic mice expressing the EPO-binding zinc finger activator polypeptide are created by microinjecting the SalI, XbaI linearised plasmid (MIEPO) containing the effector gene, into the pronuclei of fertilised eggs and re-implanting the eggs into a female mouse, as described in example 2. Progeny are screened by Southern analysis and standard PCR techniques to select the correct transgenic mice. Thymocytes and T-cells are then isolated from mice containing the zinc finger activator polypeptide, and negative control mice, according to standard surgical techniques.

The expression of the zinc finger polypeptide can be analysed, as before, using standard Western blotting and immunohistochemical procedures, using an anti-c-myc antibody.

The level of mouse EPO mRNA is assayed by standard procedures of RT-PCR and Northern blotting, using mouse EPO sequence specific primers and probes, created as explained in Example 4, to determine the amount of transcriptional activity from the endogenous EPO gene.

Increased EPO levels in the blood stream cause a concomitant rise in the number of red blood cells in an animal (Regulier, E. et al., Gene Ther. 5: 1014-1022 (1998)). Therefore, instead of, or in addition to the detection of EPO by RT-PCR, levels of EPO can be determined by measuring the number of red cells (hematocrit) in the blood of transfected mice. Blood is collected from anesthetised mice at specific time intervals into heparinised microhematocrit tubes. The hemoglobin concentration was determined by spectroscopic measurements of the cyanmet derivative. Hematocrit was determined by centrifugation in a micro-hematocrit centrifuge. Further blood analyses can be performed according to Brugnara, C. et al., Science, 232: 388-390 (1986), Trudel, M. et al., Blood, 84: 3189-3197 (1994), De Franceschi, L. et al., Blood, 94: 4307-4313 (1999), and Danon, D. & Marikovsky, Y. J. Lab. Clin. Med. 64: 668-674 (1964).

Each of the applications and patents mentioned above, and each document cited or referenced in each of the foregoing applications and patents, including during the prosecution of each of the foregoing applications and patents (ìapplication cited documentsî) and any manufacturerís instructions or catalogues for any products cited or mentioned in each of the foregoing applications and patents and in any of the application cited documents, are hereby incorporated herein by reference.

Furthermore, all documents cited in this text, and all documents cited or referenced in documents cited in this text, and any manufacturerís instructions or catalogues for any products cited or mentioned in this text, are hereby incorporated herein by reference. In particular, we hereby incorporate by reference International Patent Application Numbers PCT/GB00/02080, PCT/GB00/02071, PCT/GB00/03765, United Kingdom Patent ApplicationNumbers GB0001582.6, GB0001578.4, and GB9912635.1 as well as U.S. Ser. No. 09/478,513, PCT/GB99/03730 (published as WO00/27878A1), U.S. application Ser. No. 09/139,672, filed Aug. 25, 1998 (now U.S. Pat. No. 6,013,453), U.S. application Ser. No. 08/793,408 (now U.S. Pat. No. 6,007,988), PCT/GB95/01949 (published as WO96/06166), U.S. Ser. No. 08/422,107, WO96/32475, WO99/47656A2, WO98/53060A1, WO98/53059A1, WO98/53058A1, WO98/53057A1, WO 00/73434, WO01/00815, and U.S. Pat. Nos. 6,013,453 and 6,007,988.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A transgenic, non-human animal comprising a heterologous, engineered nucleic acid binding polypeptide which binds to a target gene and modulates its expression, in which the heterologous nucleic acid binding polypeptide is encoded by a transgene that is stably integrated into the genome of the animal, and in which the expression of a target gene in at least one cell is modulated compared to a non-transgenic animal.

2. (canceled)

3. A transgenic non-human animal according to claim 1, in which the expression of an endogenous gene is modulated.

4. A transgenic non-human animal according to claim 1, in which the gene whose expression is modulated comprises a heterologous gene which is introduced into the cell or an ancestor of that cell.

5. A transgenic non-human animal according to claim 1, in which the nucleic acid binding polypeptide binds to a promoter or other control sequence of a gene to modulate its expression.

6. A transgenic non-human animal according to claim 1, in which the gene whose expression is modulated comprises erythropoietin (EPO) or TNF receptor 1 (TNFR1).

7. A transgenic non-human animal according to claim 1, in which modulation of expression of the gene occurs in a subset of cells of the transgenic animal.

8. A transgenic non-human animal according to claim 7, in which the subset of cells comprises cells of a similar tissue type, location or developmental stage.

9. A transgenic non-human animal according to claim 1, in which modulation of expression of the gene occurs in substantially all cells of the transgenic animal.

10. A transgenic non-human animal according to claim 1, in which the nucleic acid binding polypeptide comprises a zinc finger polypeptide.

11. A transgenic non-human animal according to claim 1, in which the nucleic acid binding polypeptide further comprises a transcriptional effector domain.

12. A transgenic non-human animal according to claim 11, in which the transcriptional effector domain comprises a transcriptional repressor domain selected from the group consisting of a KRAB-A domain, an engrailed domain and a snag domain.

13. A transgenic non-human animal according to claim 11, in which the transcriptional effector domain comprises a transcriptional activation domain selected from the group consisting of VP16, VP64, transactivation domain I of the p65 subunit (ReIA) of nuclear factor-KB, transactivation domain 2 of the p65 subunit (RelA) of nuclear factor-KB, and the activation domain of CTCF.

14-16. (canceled)

17. A transgenic non-human animal according to claim 12, in which expression of the target gene in at least one cell is downregulated by at least 80% compared to a non-transgenic animal.

18-20. (canceled)

21. A method of determining the function of a gene, the method comprising the steps of
(a) providing a transgenic animal according to claim 1; and
(b) observing a phenotype of the transgenic animal as compared to an animal not comprising the transgene, thereby determining the function of the target gene.

22-24. (canceled)

25. A method of identifying a molecule which modulates the interaction between a nucleic acid binding polypeptide and a target nucleic acid sequence, the method comprising the steps of
(a) providing a transgenic animal according to claim 1;
(b) exposing the transgenic animal to a candidate molecule; and
(c) detecting binding or modulation of binding between the nucleic acid binding polypeptide and the target nucleic acid sequence.

26. A method according to claim 25, in which binding between the nucleic acid binding polypeptide and the target nucleic acid sequence is detected by detecting expression of the target nucleic acid sequence, or by detecting expression of a nucleic acid sequence linked to the target nucleic acid sequence.

27. A method according to claim 25, in which binding between the nucleic acid binding polypeptide and the target nucleic acid sequence is detected by observing a visible phenotype.

28-29. (canceled)

30. A method of producing a polypeptide, the method comprising the steps of
(a) providing a transgenic animal according to claim 1, wherein the target gene is the polypeptide and further wherein the nucleic acid binding polypeptide up-regulates the expression of the polypeptide; and
(b) harvesting the polypeptide from the transgenic animal.

31. A method according to claim 30, in which the polypeptide is secreted into the mammary or other fluid of the animal, and in which the polypeptide is isolated from the fluid.

32. A polypeptide produced by a method according to claim 30.
